(19) United States
(12) Patent Application Publication
(10) Pub. No.: US 2026/0102262 A1
(43) Pub. Date: Apr. 16, 2026

(54) Multi-Task Hand Support Device with Biological Signal Acquisition for Operation with or without Bionic Limbs, Prosthetics and Osseointegration Limbs Implants, including Exoskeletons Wearables

(71) Applicant: Jesus Perea-OcHoa, Montebello,, CA (US)

(72) Inventor: Jesus Perea-OcHoa, Montebello,, CA (US)

(21) Appl. No.: 19/354,843

(22) Filed: Oct. 10, 2025

Related U.S. Application Data
(60) Provisional application No. 63/706,643, filed on Oct. 12, 2024.

(51) Int. Cl. A61F 2/54; A61F 2/50; A61F 2/68; A61F 2/70; A61F 2/72; A61F 2/76; A61F 2/78

(52) U.S. Cl. CPC A61F 2/54; A61F 2/72; A61F 2/7812; A61F 2002/502; A61F 2002/5061; A61F 2002/689; A61F 2002/701; A61F 2002/704; A61F 2002/707; A61F 2002/769; A61F 2002/7837

(57) ABSTRACT

Multi-Task Hand Support Device with Biological Signal Acquisition for Operation with or without Bionic Limbs, Prosthetics and Osseointegration Limbs Implants, including Exoskeletons Wearables A hand support device for an input apparatus, such as typing apparatus like keyboard and touch screen panel, any smart electronic device and/or built-in system thereof, is configured for input apparatus like keyboard, headset, laptop or other communication computer input apparatus, or gaming device, including any smart glasses/goggle VR, AR, MR, XR (AI) program display medium headset for the purpose of the user supporting his or her uppers extremities, in particular hands and wrist during repetitive task and or for prolong cervical spine, thoracic spine, and lumbar spine muscular posture tension, in a wrist support typing posture for multitasking, including upper extremities neuroplasticity injuries, through imagined, performed or partially performed EMG, EEG and or EcoG signal acquisition systems.

### CROSS REFERENCE OF RELATED APPLICATION

This application is a non-provisional application that claims the benefit of priority under 35 U.S.C. § 119 to a provisional application, application number 63/706,643, filed Oct. 12, 2024, which is incorporated herewith by references in its entity.

### BACKGROUND OF THE PRESENT INVENTION

### Field of Invention

The present invention relates to a hand support device for an input apparatus, such as typing apparatus like keyboard and touch screen panel, laptop accessory and/or built-in system thereof, and more particularly a hand support device, adapted for operating in a multi-task interactive system with or without bionic limbs, prosthetics, modular prosthetic limbs. Furthermore, the hand support device provides posture support while working with input apparatus like keyboard, headset, laptop or other communication computer input apparatus, or gaming device, including augmented reality devices (AR), virtual reality devices (VR), mix reality (MR) and extended reality (XR) for the purpose of the user supporting his or her uppers extremities, in particular hands and wrist during repetitive task and or for prolong cervicothoracic spine muscular posture tension, in bilateral upper extremity support posture, including neuroplasticity injuries.

### Description of Related Arts

Upper extremity amputees and missing limb deformities require prosthetics and osseointegration limbs implants, including targeted muscle reinnervation and targeted sensory reinnervation. Neuroplasticity patients have a reduction in motion from reduction or lack of communication between the brain nerve functions and the muscle function, due to various illnesses like stroke, brachial plexus injury, brain injury, spine cord injury and other neurological diseases. In either case amputation, congenital deformity, or neuroplasticity they all lack the bilateral upper extremities support. The invention teaches the technological interoperability among inputting systems with the hand support device to reduce the dexterity demands for advanced computer inputting capacity.

Almost all people work on the computer to type, like editing codes, writing reports, sending emails or accessing information, etc. In other words, temporary and permanent upper extremities injuries of any type that are disabling in nature affect computer operations. The reports say that people are sitting an average of 9 hours a day, 60% of which are at work facing the computer, which is harmful to the health of people, especially when people sit in the wrong position. All computing devices like laptops, keyboard computer gaming devices, (AV/AR) devices, communication devices, require a particular level of human's physical effort and stamina to produce a task and for enjoyment.

### SUMMARY OF THE PRESENT INVENTION

The invention is advantageous in that it provides a multi-task hand support device with signal acquisition for input apparatus, such as typing apparatus like keyboard and touch screen panel, laptop accessory and/or built-in system thereof, and any computer operation with or without bionic limbs and prosthetics with or without osseointegration limbs implants, including orthotics and exoskeletons wearables.

Furthermore, the hand support device provides posture support while working with input apparatus like keyboard, headset, laptop or other communication computer input apparatus, or gaming device, including augmented reality devices (AR) and/or virtual reality devices (VR) for the purpose of the user supporting his or her uppers extremities, in particular hands and wrist during repetitive task and or for prolong cervicothoracic spine muscular posture tension, in a wrist support typing posture, including neuroplasticity injuries

The invention teaches a novel approach to operating computers, for example but not limited to “Human machine interphase that can connect to the prosthesis, orthotics and osseointegration limbs implants, with or without a hand support device, with or without exoskeleton, with or without inputting wearables. Within a physical medium or a non-physical medium operating with or without sideline programs, with or without gestures “grip like movements”, acquiring signal acquisition for recording and for interchanging signal acquisition for programming through Artificial Intelligence learning, deep learning or machine learning. While providing additional physical support to both upper extremities for optimum posture alignment.

Additionally, upper extremity disability limits prolong sitting and typing due to poor posture. Prosthetics, exoskeletons, orthotics devices and osseointegration titanium limb implants need to withstand muscle fatigue due to gravitational forces and counter pressure without a hand support device. The current chair and desk industry lack the support for various individual sizes in relation to the desk position and chair position. In other words, the chair may be adjustable, but the table is fixed and vice versa. Furthermore, the chair and table are sold and built independently of each other. In other words, the manufacturing process does not teach the creation of a synergic system between the chair, the hand support device and table.

Presently, most people with missing limbs or Neuroplasticity have limited computer productivity, that is further exacerbated from poor posture compounding their disability. People with missing limbs or Neuroplasticity have no choice and are forced to endure painful posture positions. Their cervical spine and thoracic vertebral in the wrong posture of forward flexion and their muscles of the posterior neck are also strained due to short or long-term poor posture that eventually leads to cervical, spine, thoracic spine and lumbar spine spondylosis and other muscular skeletal injuries. At the same time, poor ergonomic sitting without posture support can cause gravity and muscle fatigue to aggravate the thoracolumbar region, wherein neurogenic, anxiogenic, vertebralgenic, discogenic and congenital transient or permanent injuries and medical conditions create poor ergonomic posture and uneven distributed between the musculoskeletal system, which causes insidious pathology like kyphosis and multiple other dorsopathy injuries etc. The continuous daily strains eventually impact other muscular skeletal systems in conjunction with the original injury or the missing limb.

It is important and necessary to maintain a correct sitting and typing position while using the computer. When we sit and type, our hands will rise high in the air over the keyboard and eventually time will produce fatigue causing the wrong siting and typing posture to manifest, especially our cervical spine and thoracic vertebras in a wrong state of posture alignment. Even if our elbows and forearms are supported, these problems will not be solved, without the correct posture on the hand support device.

In addition to the hunchback and the cervical spondylosis, the poor sitting and typing posture also causes thoracic enthesopathy, upper crossed syndrome which manifests as inhibited neck flexors, inhibited rhomboids and serratus anterior, tight deltoid and tight upper trapezius and levator scapulae muscles, and tension headache can all manifest with or without (AR, VR, MX, XR) devices.

In addition, most electronic devices are powerful enough to run two applications at the same time for multi-tasking. For example, the player is able to play via a game application and chat with friends via another chat application. However, these applications are controlled by one single input. In other words, when the player wants to control the game, he or she must switch the input corresponding to the game application. When the player wants to chat with friends, he or she must switch the input back to the chat application from the game application. This switching manner will interfere the flow of the game and the chatting as well in social media computer operations.

Such switching burden also exists in processing software applications and/or computer languages, such as processing a word or graphic document, by opening one window and chatting with friends with a communication software or APP by opening another window. The user is required to switch between the windows from time to time too. Specially, in users with physical medical conditions, it can be severely limiting to operate programs like these that require continues dexterity with or without any hand therapeutic support device.

Presently, there are no hand support devices with electronic components to intergrade with bionic limbs, prosthetics, osseointegration limbs implants and orthotics, including exoskeletons systems. It is important to mention that the hand support device can interface with the targeted muscle reinnervation and targeted sensory reinnervation or other similar surgical operations to enable the operator to multitask with limited dexterity or tactile sensory. In other the hand support device has the physical electronic hardware and operates the software to interface with external displays, AR, VR, MR, XR, holograms, remote computers, portable computers, smart electronic, smart wearables, supper computers or quantum computers. The hand support device can also be an additional source for re-charging wirely or wireless any bionic limbs, prosthetics, osseointegration limbs implants and orthotics, including exoskeletons, etc. Furthermore, the hand support device can be portable, foldable, and/or collapsible carry-on. Allowing for computer operations on demand. The invention teaches a portable hand support device that can be independently utilized with or without the brief case carry-on accessory. Additionally, the hand support device briefcase carry-on accessory can be built into a portable table that adjusts to the consumers requirements, on demand.

The invention teaches a hand support device that operates with any biological neuromuscular signal acquisition, like EMG, including (EcoG implants) with or without bionic limbs, prosthetics, osseointegration limbs implants and orthotics, including exoskeletons wearables for interacting with any computer and to navigate any computer. The hand support devices provide bilateral hand support with or without bionic limbs, prosthetics, osseointegration limbs implants and orthotics, including exoskeletons. The hand support device provides the proper posture for good kinetic mechanics for the user with or without bionic limbs, prosthetics, osseointegration limbs implants and orthotics, including exoskeletons, etc. Additionally, the hand support device interfaces the user within the digital computer and quantum computers. Allowing for scalability of biological signal acquisition in various options and for recording, programming while inputting within any computing medium, including (AR, VR, MX, XR). In other words, reducing dexterity requirements and physical tactile demands for any user with or without robotic limbs bionic limbs, prosthetics, osseointegration limbs implants and orthotics, including exoskeletons systems, etc.

It is important to mention that the hand support device may or may not have one or more built-in computer electronic (processor), may or may not operate with external server processor, in either case the hand support device has the capacity of interfacing with other computer systems including any bionic limbs, prosthetics, osseointegration limbs implants and orthotics, including exoskeletons, etc. The hand support device is the physical bridge and digital bridge of the bionic limbs, prosthetics, osseointegration limbs implants and orthotics, including exoskeletons systems, for controlling and operating within any digital medium. Furthermore, the hand support device artificial intelligence program records all imagined performed or partially movements intentional and non-intentional biological signals acquisition algorithm computations to aid the user through injuries, fatigue, or any other transient or permanent illness, for operating computer devices. The invention encourages proper kinetic movements with or without bionic limbs, prosthetics, osseointegration limbs implants and orthotics, including exoskeletons, etc.. Additionally, the hand support device provides the support for scalable signal acquisition inputting during computer operation with or without one or more bionic limbs, prosthetics, osseointegration limbs implants and orthotics, including exoskeletons, etc. Furthermore, the same action point, or similar touch points, kinetic movements, grasping with various speeds and strengths are scalable gestures for interfacing with any computer electronic device with or without bionic limbs, prosthetics, osseointegration limbs implants and orthotics, including exoskeletons with or without their corresponding physical movements. The invention teaches any external or internal biological signal acquisition operating with the hand support device.

Additionally, the hand support device can be recharged form traditional electrical wall outlets, solar charging systems, or other power generator systems. The hand support device provides recharging the battery electrical energy of one or more bionic limbs, prosthetics, osseointegration limbs implants and orthotics, including exoskeletons systems, while the bionic limbs, prosthetics, orthotics and exoskeletons systems (wearable) conserved energy in the dormant state. The hand support device can automatically active the dormant state automatically or the user activates this control. The dormant state of the bionic limbs, prosthetics, osseointegration limb implants and orthotics, including exoskeletons is a function of the hardware and software working together to turn on or off the physical movements of one or more bionic limbs, prosthetics, osseointegration limbs implants and orthotics, including exoskeletons systems, while the bionic limbs, prosthetics, orthotics and exoskeletons systems (wearable) swish to EMG, EEG and or EcoG signal acquisition (AI) program interface without any physical movements of the bionic limbs, prosthetics, osseointegration limbs implants and orthotics, including exoskeletons, for operating other computer devices like traditional computers, smart glasses/goggle VR, AR, MR, XR (AI) program devices. In other words, any external sensor or internal biological signal acquisition previously used to operate movement of any limbs, prosthetics, osseointegration limbs implants and orthotics, including exoskeletons are change/converted to EMG, EEG and or EcoG signal acquisition (AI) program interface to operated digital gestures, digital functions for controlling and operating computing operations with or without any movement of the physical device of the bionic limbs, prosthetics, osseointegration limbs implants and orthotics, including exoskeletons systems devices.

Furthermore, any external or internal biological signal required by the mechanical system for movement of the bionic limbs, prosthetics, osseointegration limbs implants and orthotics, including exoskeletons can be altered, switched, changed, and converted, to interface for control computer digital computer inputting with or without producing any movement within one or more bionic limbs, prosthetics, osseointegration limbs implants and orthotics, including exoskeletons devices. It is important to mention the dormant state is temporary, while the user is operating computer systems. Furthermore, the dormant state can be control by the user and can be disable by the user or limited by the user or the selected program can have pre-selected limitations pre-program by the user. Preferably, in the dormant state of the bionic limbs, prosthetics, osseointegration limbs implants and orthotics, including exoskeletons systems wearable are operational near the hand support device to facilitate multitasking operations of other executive computing devices. For example, the user of an upper bionic limb, prosthetic, osseointegration limb implant and orthotic, including exoskeleton system wearable can hold a smart phone without additional mechanical movement from the bionic limb, prosthetic, osseointegration limb implant and orthotic, including exoskeleton system wearable. Thereby, the primary function of the EMG, EEG and or EcoG signal acquisition (AI) program form operating the motion of the bionic limb, prosthetic, osseointegration limb implant and orthotic, including exoskeleton system wearable are switched to operate other interface computer executive programs without producing mechanical motion of the bionic limb, prosthetic, osseointegration limb implant and orthotic, including exoskeleton system wearable.

The development of external or internal biological signal acquisition technology can be used to operate movement of bionic limbs, prosthetics, osseointegration limbs implants and orthotics wearables, including exoskeletons, resulting in interface physical and operational functions where the user is not required to physically perform the task to produce the desire outcome. For example, any external or internal biological signal acquisition previously used to operate movement the bionic limbs, prosthetics, osseointegration limbs implants and orthotics, including exoskeletons can interface with the car door and the car door opens automatically without the user physically doing the task, etc.

Primarily, the hand support device provides conservation of energy (sleep mode or dormant) within one or both bionic limbs, prosthetics, osseointegration limbs implants and orthotics, including exoskeletons wearables, including one or both non-biological hands. The invention teaches that the hand support device hardware and software have synergistic benefits for non-biological limbs and biological limbs. In either case, when missing one upper extremity or missing both upper extremities the hand support device provides bilateral upper extremity support on one or more bionic limbs, prosthetics, osseointegration limbs implants and orthotics, including exoskeletons wearables thereby providing cervical spine, thoracic spine and lumbar spine proper alignment. It is important to mention that the (sleep mode or dormant state) is for complete or partial stoppage of motion of the bionic limbs, prosthetics, osseointegration limbs implants and orthotics, including exoskeleton wearable devices. For example, bionic limbs, prosthetics, osseointegration limbs implants and orthotics, including exoskeleton devices when position on the hand support device may or may not stop all the movements of all the fingers, hand wrist or elbow. In another example, the index finger and the third finger are the only two finger with mechanical motion for producing gestures on a touch screen control or for gesture production with or without a touch screen control, or for camera hand recognition programs or sign language recognition programs to operate computers through a non-physical medium or a combination of tactile imputing (biological hand) and non-tactile imputing (non-biological hand). Alternatively, the one or more bionic limbs, prosthetics, osseointegration limbs implants and orthotics, including exoskeletons may continue to have full motion control for various grips, articulation positions, joint movements and various hinge angles (AI robotic sign language recognition). In other words, the camera in the hand support device captures the physically performed grip/gesture to generate a computer response. Alternatively, the hand support device captures the same signal acquisition EMG, EEG, and ECoG intended for movement of the bionic limbs, prosthetics, osseointegration limbs implants and orthotics, including exoskeleton and interfaces with other computers enables the EMG, EEG and or EcoG signal acquisition to produces computer control to navigate digital programs with or without the robotic movement of the non-biological limb or without the manual movement of the biological limb to performed grips or gesture controls. Through signal acquisition the biological hand or non-biological hand can generate computer inputting capabilities with partial movements to no movement of the non-biological limb (robotic movement) or of the biological limb (grip/gestures). In other words, partial movements for the non-biological hand and the biological hand produces conservation of energy in either case, in the sleep mode or in the dormant state.

Furthermore, the bionic limbs, prosthetics, osseointegration limbs implants and orthotics, including exoskeleton wearables devices may or may not continue to have all the movements within stander operations, including enhanced speed or joint rotational alteration. In other words, the speed of the bionic limbs, prosthetics, osseointegration limbs implants and orthotics, including exoskeleton devices may or may not be augmented along and the bionic joints rotational capacity for the purpose of computer operations. For example, in the joint rotational activation the fingers, wrist etc. can have additional angular rotations for the purpose of multitasking, for example the finger can rotated at the MCP joint 180-360 degree or the PIP joint rotated 180-360 degree to hold a smart phone. Additionally the strength (applied pressure) capacity in the bionic limbs, prosthetics, osseointegration limbs implants and orthotics, including exoskeleton wearables devices can be used as a control gage/intensity control for operating computer work, for example the strength apply to the pinching of two finger (pressure sensors) performed or imaged can increase the volume or brightness of a program according to the user pre-program. It is important to mention that the strength control comes from the EMG, EEG, EcoG signal acquisition and therefore may or may not have external physical pressure sensors in the biological upper extremities or in the bionic limbs, prosthetics, osseointegration limbs implants and orthotics, including exoskeleton wearables devices that can also be used control other computer functions within the biological upper extremities or within non-biological upper extremities. Alternatively, the bionic limbs, prosthetics, osseointegration limbs implants and orthotics, including exoskeleton devices strength mechanical capacity may or may not be reduced when interface with the hand support device, for the purpose of computer operations.

The EMG, EEG and EcoG sensors, the signal acquisition, the Artificial Intelligence, the recordings and computer programing may or may not be dependent on targeted muscle innervation surgery and or targeted sensory innervation surgery. Furthermore, either surgery independently or together can have signal acquisition to interfacing with computer devices for operations with or without bionic limbs, prosthetics, osseointegration limbs implants and orthotics, including exoskeleton wearables. It is important to mention that other computer electronic devices interface function may or may not be depending on the hand support device computer software compatibility relation. It is important to mention that the hand support device interface function may or may not depend on the computer software compatibility in-relation with the bionic limbs, prosthetics, osseointegration limbs implants and orthotics, including exoskeleton for operating other computer electronic devices. These are examples and are not limitations to the various options that the hand support device provides to the user with or without bionic limbs, prosthetics, osseointegration limbs implants and orthotics, including exoskeleton devices, etc. Additionally, the hand support device, the bionic limbs, prosthetics, osseointegration limbs implants and orthotics, including exoskeleton wearables along with the signal acquisition capacity utilize AI inference.

The hand support device will have additional EMG and EEG sensors with or without bionic limbs, prosthetics, orthotics and including exoskeleton. The additional EMG, and EEG sensors may or may not be used to operate the bionic limbs, prosthetics, orthotics and including exoskeleton systems. The additional EMG sensors and EEG sensors within hand support device allows for additional placements of EMG sensors and EGG sensors enabling multiple other EMG sensors or EEG sensors for additional signal acquisition along other anatomical neuromuscular locations for signal acquisition for scalability and for a more robust signal acquisition, with or without wearable amplifiers from a stander skin sensors in addition to the bionic limbs, prosthetics, osseointegration limbs implants and orthotics, exoskeleton, including other wearable devices, for interfacing and inputting programing computer control options. The hand support device ensures that the upper extremity supports the proper posture for enabling scalability of the precise EMG/EGG signal acquisition without or with limited random unwanted signal acquisition noise, created by fatigue or poor posture muscle activation. Therefore, the hand support device enables precision combination of EMG/EEG signal acquisitions to be scaled and or complex collections of EMG/EEG signal acquisition to increase multitasking capacity through AI inference. Furthermore, the EMG/EEG neuromuscular sensors signal acquisition are acquired and recorded from either upper extremity, from both upper extremities, at the same time or in a sequential manner, from bilateral biological upper extremities or from one or more bionic limbs, prosthetics, osseointegration limbs implants and orthotics, exoskeleton, including other wearable devices.

It is important to mention the hand support device provides the means for meaningful (precision recording) and programing between the user and signal acquisition/Artificial intelligence algorithms program (AI inference) for scalable programming. The upper extremity muscles and nerves must have a neutral baseline positioning from which the signal acquisition originates corresponding to the muscle and nerves EMG signals, including EEG signal acquisition for recording and learning within Deep Learning artificial intelligence (Machine Learning) to generate the corrected computer inputting in traditional computers and within quantum computers. For example, consider multiple EMG sensors for signal acquisition position in multiple muscles groups in one or both upper extremities, each sensor signal acquisition generates an independent computer response. Alternatively, various combinations of muscle and nerve activations produces the scalable signal acquisition for independent or co-dependent computer responses. Additionally, the hand support device provides the user the upper extremity support for proper signal acquisition within EMG sensors and or EEG sensors collectively and independently, or at the same time. In other words, the user without proper posture alignment, without a hand support device is prone to produce erroneous EMG and EEG signal acquisition for controlling any computer electronic device, with or without bionic limbs, prosthetics, osseointegration limbs implants and orthotics, including exoskeletons wearables. It is important to mention that the EMG and EEG signal acquisition where mainly mentioned for illustration purposes and that everything in relation to nerve signals acquisition also applies to the EcoG sensors system.

Operating multi-task interactive electronic devices may also be executed through gesture operation with or without an inputting apparatus and will initially require a hand support device. For example, the most commonly used gesture in operating a program on a display screen is, the zoom-in gesture and zoom-out gesture. The zoom-in and zoom-out (pinching-in and pinching-out gestures) on the display screen can respectively help to zoom in and zoom out an object, such as a picture, a document or a figure, displayed on the screen which can be the same display screen or another displaying screen. The zoom-in gesture or the zoom-out gesture is simply executed through two fingers of a user touching, producing gesture a complete gesture, a partial gesture and or imagine gesture EMG, EEG, EcoG signal acquisition for inputting within the physical display screen medium, or within a non-physical medium electronic computer device (AR, VR, MX, and XR). Furthermore, the invention teaches the hand support device in operations with other electronic computer devices smart phones, smart glasses etc., in combination interoperability with multiple inputting systems, like (AR, VR, MX, and XR).

In other words, the hand support device houses one or more processer and or interfaces with external servers to activate the physical inputting system and/or the signal acquisition inputting system from one or more biological upper extremities and/or from one or more non-biological upper extremities for the purpose of operating a touch screen or a keyboard, etc. The combination of the hand support device activating the keyboard or touch screen also activates the sideline program. The combination of the hand support device, keyboard, touch screen, and sideline program also activates the (AR, VR, MX, and XR), including Therefore, the user can develop a single eye tracking movement, a gesture or a voice command (imagine or performed) for a program, for a set of programs in VR, AR, MR, XR and or holograms to navigate the monotonous (step by step) computer work.

Furthermore, the invention teaches displaying and controlling two or more programs, icons, displays, functions, or applications, with the least adjustment, physical demand or manual dexterity. Wherein, the visible sideline mode separates the desired programs, icons, displays, functions, or applications within a VR, VA, MR, XR, or hologram medium. Accomplishing the workload reduction through machine learning, deep learning, AI inference or any other algorithmic calculation process independent or in combination through artificial intelligence. AI inference programs applicable to performed or imagined gesture, eye movement or voice command, or in combination with other inputting systems dependent or independent of EMG, EEG and or EcoG signal acquisition for interfacing with other electronic devices through the hand support device for precision signal acquisition. Wherein a single or combination of gesture, eye motion or voice command performed or imagined can generate the desired program task, or functions. Then the step-by-step repetitiveness demands in the present computer workflow can be replaced, eliminated or reduced the reparative eye tracking movements, gestures or voice commands in order to get to the desired output. Therefore, reducing work time and increasing productivity. For example, the eyeball tracking program may or may not be the main inputting modality independent or in combination with gestures, motion sensors, sign language programs, voice commands programs. Additionally, any inputting system may be designated primary for independent usage or in combination with other inputting devices and programs. Any inputting system may have shared redundant inputting capacities with other performed or imagined inputting systems as a means for a more ergonomic and robust inputting system through the hand support device.

Currently in the market, there is no organization for program operating gesture that can control the operating program while inputting at the same time with multiple inputting systems. However, it is a great demand to the user for operating one or more programs and/or on one or more display screens of an electronic device such as a notebook computer, a tablet, a smart phone, or a slot machine. However, multiple programs or applications are developed and installed in an electronic device and two or more programs or applications (APPs) are able to be opened at the same time to function in two or more different windows but, generally, only one program is operational for physical control while the other programs or applications may merely display without the capacity to be operated at the same time. These programs or applications share a single input device (one to one input capacity), that is only one inputting can control one of the display screen programs. The user is forced to shift the physical activities of inputting between two programs or APPs, that substantially increases the workload by having to alternate from one program to the other program, decreases productivity with numerous (back and forth) physical control movements for each separated program, and increases time consumption when multitasking under current operation system. In other words, the mere input device only controls one primary display program or only one primary display application at one time.

With the development of metaverse (meta universe/simulate environment), virtual reality (VR), mixed reality (MR), extended reality (XR), and augmented reality (AR) technologies are able to be interacted with the reality world through the VR, MR, XR or AR apparatus that the virtual reality and the reality, the reality and the augmented reality, or multiple virtual realities are being displayed at the same time via a displaying apparatus (such as VR/AR/XR/MR smart glasses or goggles glasses) etc. The input apparatus, such as touch screen panel, keyboard, mouse pad, etc., are capable of configured to allow the user to operate the multi-task interoperable electronic devices with the input apparatus with gestures performed of imagined. It is important to mention AI inference programs require the initial inputting of the data programs, executive programs, signal acquisition recordings, display recordings in real time for executing a designated task in every multiple variation.

When a user has to continue to work on the inputting apparatus, even simply gestures to operate the multi-task interactive electronic devices, the user still needs to withstand muscle, wrists and finger joints fatigue due to gravitational force, including cervical spine, thoracic spine and lumbar spine pathology. There is an urge demand for an effective hand support device for operating multi-task interactive electronic devices and system for displaying sideline operation to all users, especially to the children, senior people and upper limb injured patients with or without bionic limbs, prosthetics, osseointegration limbs implants and orthotics, including exoskeletons wearable systems.

Another advantage of the invention is to provide a portable or fix hand support device for input apparatus, such as a keyboard, mouse, mouse pad, touch panel, laptop, notebook, or the likes, for operating electronic devices, multi-task interactive electronic devices with or without displaying sideline operation, which provides assistance and support for a user to rest both hands and wrists or one or more prosthetics, so that the user needs less physical effort while inputting computer work with or without bionic limbs, prosthetics, osseointegration limbs implants and orthotics, including exoskeletons.

The invention is also advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operation, which provides assistance and support for a user to rest both hands and wrists, so that the user needs less efforts while inputting computer work with or without bionic limbs, prosthetics, osseointegration limbs implants and orthotics, including exoskeletons and at the same time electrically re-charging the bionic limbs, prosthetics, osseointegration limbs implants and orthotics, including exoskeletons systems through the hand support device.

The invention is also advantageous in that it provides a portable or fix hand support device for inputting apparatus, such as a keyboard, mouse, mouse pad, touch panel, laptop, notebook, or the likes, for operating electronic devices, multi-task interactive electronic devices with or without displaying sideline operation, while wirely or wireless electrically charging any bionic limbs, prosthetics, osseointegration limbs implants and orthotics, including exoskeletons.

The invention is also advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operation, while wirely or wireless electrically charging one or more computer electronic device.

The invention is also advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operation, while wirely or wireless electrically charging one or more electronic device, including one or more bionic limbs, prosthetics, osseointegration limbs implants and orthotics, including exoskeletons.

The invention is also advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operation, while wirely or wireless electrically charging one or more electronic device, at the same time with one or more bionic limbs, prosthetics, osseointegration limbs implants and orthotics, including exoskeletons. With the option to have (sleep mode or dormant state) that is for complete or partial motion restriction of the bionic limbs, prosthetics, osseointegration limbs implants and orthotics, including exoskeleton devices.

The invention is also advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operation, while wirely or wireless electrically charging one or more electronic device, at the same time one or more bionic limbs, prosthetics, osseointegration limbs implants and orthotics, including exoskeletons. With the option to have (sleep mode or dormant state) that is for complete or partial motion restriction of the bionic limbs, prosthetics, osseointegration limbs implants and orthotics, including exoskeleton devices. Wherein, various EMG, EEG and or EcoG signal acquisitions are used to interface for controlling various computer electronic device functions at the same time wireless or wirely.

The invention is also advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operation, while wirely or wireless electrically charging one or more electronic device, at the same time one or more bionic limbs, prosthetics, osseointegration limbs implants and orthotics, including exoskeletons. With the option to have (sleep mode or dormant state) that is for complete or partial motion restriction of the bionic limbs, prosthetics, osseointegration limbs implants and orthotics, including exoskeleton devices.

The invention is also advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operations, wirely or wireless electrically charging one or more electronic device, at the same time while one or more signal acquisition EMG, EEG and or EcoG signal acquisition sensors are operational.

The invention is also advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operations, wirely or wireless electrically charging one or more electronic device, at the same time while one or more signal acquisition EMG, EEG and or EcoG signal acquisition sensors are operational.

The invention is also advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operations, wirely or wireless electrically charging one or more electronic device, at the same time while one or more signal acquisition from the EMG, EEG and or EcoG signal acquisition sensors are operational.

The invention is also advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operations, wirely or wireless electrically charging one or more electronic device, at the same time while one or more signal acquisition from the EMG, EEG and or EcoG signal acquisition sensors are operational. Wherein, various EMG and EEG/EcoG signal acquisitions are interface for controlling various computers within one or more interface computer electronic device, in a timely manner and or at the same time.

The invention is advantageous in that it provides a portable or fix hand support device for input apparatus, such as a keyboard, mouse, mouse pad, touch panel, laptop, notebook, or the likes, for operating electronic devices, multi-task interactive electronic devices with or without displaying sideline operation, while wirely or wireless electrically charging one or more electronic device, at the same time while operating one or more bionic limbs, prosthetics, osseointegration limbs implants and orthotics, including exoskeletons devices, with the option to have (sleep mode or dormant state) for complete or partial motion restriction of the bionic limbs, prosthetics, osseointegration limbs implants and orthotics, including exoskeleton devices. Wherein, various EMG and EGG/EcoG signal acquisitions are interface for controlling various computer sideline programs within one or more computer electronic devices at the same time within VR/AR/XR/MR smart glasses or goggles glasses. The invention teaches the eye movement program, eye tracking system program, line of sight program, and eye blinking program are used to navigate in any computer program display and control VR, AR, MR, XR interoperability within a 2D-dimensional touch screen work environment and a 3D-dimensional inputting computer medium. The invention teaches the eye movement, eye tracking system, line of sight, eye blinking, gestures, EMG, EEG and or EcoG signal acquisition internal and external motion sensors and voice command are used to navigate in a program display and control VR, AR, MR, XR in a D2-dimensional work environment.

Alternatively, the EMG, EEG and or EcoG signal acquisition (AI) program can be used to operate VR, AR, MR, XR devices smart glasses/goggles with artificial intelligence programs. The invention also teaches one or more (AI) programs operating in the same program or in separate programs with separate functions. It is important to mention EMG, EEG and or EcoG signal acquisition (AI) programs can interface with VR, AR, MR and XR programs for controlling digital medium within the VR, AR, MR and XR. The invention also teaches VR, AR, MR, XR programs and VR, AR, MR, XR devices that can interface to operate and control traditional computer devices 2D-dimensional or 3D-dimensional holograms programs. Furthermore, EMG, EEG and or EcoG signal acquisition (AI) programs can interface with traditional computer devices 2D-dimensional or 3D-dimensional holograms programs independently or collectively with smart glasses/goggle artificial intelligence programs. In other words, with any smart glasses/goggles with or without EMG, EEG and or EcoG signal acquisition (AI) programs, the user can interface and operate traditional computers or 3D-dimensional holograms programs. For example, the user with smart glasses/goggles may bend the head with VR, AR, MR, XR devices smart glasses/goggles artificial intelligence programs to the right and that will move the traditional cursor to the right in a traditional computer wirely, wireless, USB port. Alternatively, the EMG, EEG and or EcoG signal acquisition may also interface with traditional computer devices 2D-dimensional or 3D-dimensional holograms programs to generate the cursor's movement through voice command, physical gesture, or eye movement signal acquisition (AI) program dependent (imagined, performed or partially performed). Furthermore, the EMG, EEG and or EcoG signal acquisition (AI) programs and the VR, AR, MR, XR digital devices (AI) can operate together at the same time in real time to control traditional computers systems. For example, the user with smart glasses/goggles (VR, AR, MR, XR) bends his head to the right and the traditional cursor in the traditional computer moves to the right. At the same time the EMG, EEG and or EcoG signal acquisition (AI) program gesture can be imagined, performed or partially performed as a (click) to activate the selected function to generate a computer response in the same traditional computer.

It is important to mention that the Artificial intelligence program is not only used for interfacing through EMG, EEG and or EcoG signal acquisition (AI) programs. The Artificial intelligence (AI) inference programs can operate independently or collectively within the smart glasses/goggles program. For example, in the abovementioned embodiment the user is moving the cursor within a traditional computer devices 2D-dimensional or 3D-dimensional holograms program through the motion of the smart glasses/goggles (AI) program and controlling the program through the EMG, EEG and or EcoG signal acquisition (AI) program gesture. In another words, the smart glasses/goggle display (AI) medium independent or collectively with the Artificial Intelligence program EMG, EEG and or EcoG signal acquisition (AI) program can display that the user, has made the incorrect selection or guide the user by displaying directions within the smart glasses/goggles display medium to produce the correct selection in the traditional computer program. Alternatively, the EMG, EEG and or EcoG signal acquisition (AI) program interface may recognize that the biological signal is being (imagined, performed or partially performed) at the incorrected icon or location for the desired program function and thereby not active the selected incorrect application. Therefore, the invention teaches one or more artificial intelligence programs operating within a smart glasses/goggle VR, AR, MR, XR (AI) program device display medium and or, one or more artificial intelligence programs operating independent or together within the EMG, EEG and or EcoG signal acquisition (AI) program interface. The invention illustrates the multiple ways that (AI) programs can operate together or independently at the same time or in alternating manner.

Additional advantages and features of the invention will become apparent from the description which follows and may be realized by means of the instrumentalities and combinations particularly point out in the appended claims.

### BRIEF DESCRIPTION OF THE DRAWINGS

FIG. 1 is a perspective view of a hand support device according to a preferred embodiment of the present invention.

FIG. 2 is a partial perspective view of the hand support device according to the above preferred embodiment of the present invention.

FIG. 3 is a schematic view of the hand support device according to the above preferred embodiment of the present invention.

FIG. 4 is an application view of the hand support device according to the above preferred embodiment of the present invention.

FIG. 5 is a schematic view illustrating the hand support device according to the above preferred embodiment of the present invention.

FIG. 6 is another schematic view illustrating the hand support device according to the above preferred embodiment of the present invention.

FIG. 7 is another schematic view illustrating the hand support device according to the above preferred embodiment of the present invention.

FIG. 8 is another schematic view illustrating the hand support device according to the above preferred embodiment of the present invention.

FIG. 9 is another schematic view illustrating the hand support device according to the above preferred embodiment of the present invention.

FIG. 10 is a schematic view of the hand support device according to the above second embodiment of the present invention.

FIG. 11 is a schematic view illustrating the hand support device according to the above second embodiment of the present invention.

FIG. 12 is a schematic view illustrating the hand support device and a cloud server according to the above second embodiment of the present invention.

FIG. 13 is a schematic view illustrating interoperable inputting systems according to the above preferred embodiment of the present invention.

FIG. 14 is a schematic view illustrating an operation of the hand support device according to the above preferred embodiment of the present invention.

FIG. 15 is a sectional schematic view illustrating the hand support device according to the above preferred embodiment of the present invention.

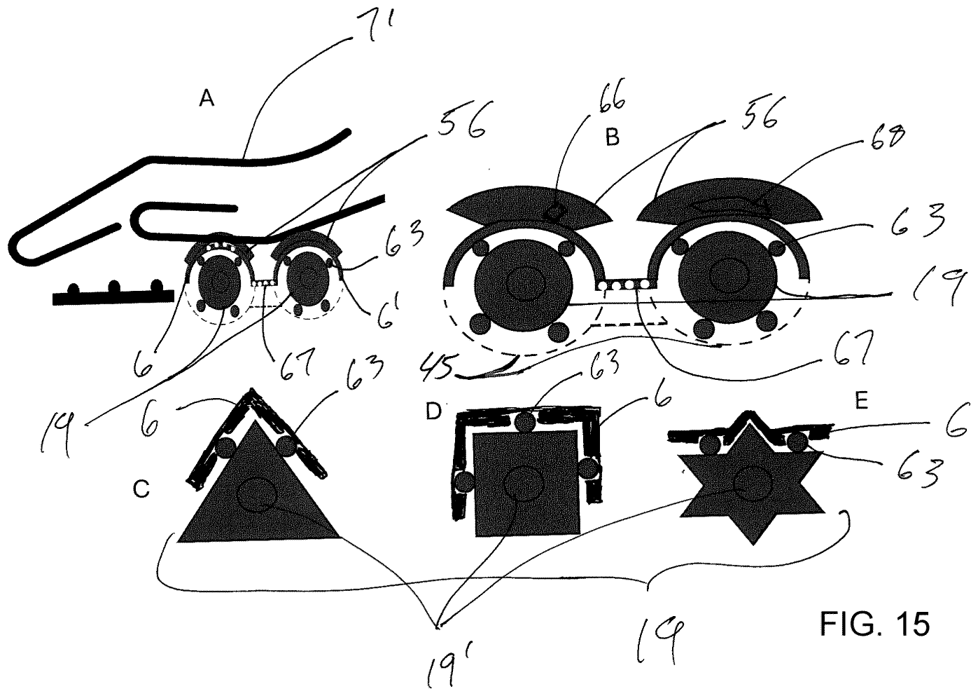

FIG. 16 is a schematic view illustrating the hand support device according to the above preferred embodiment of the present invention.

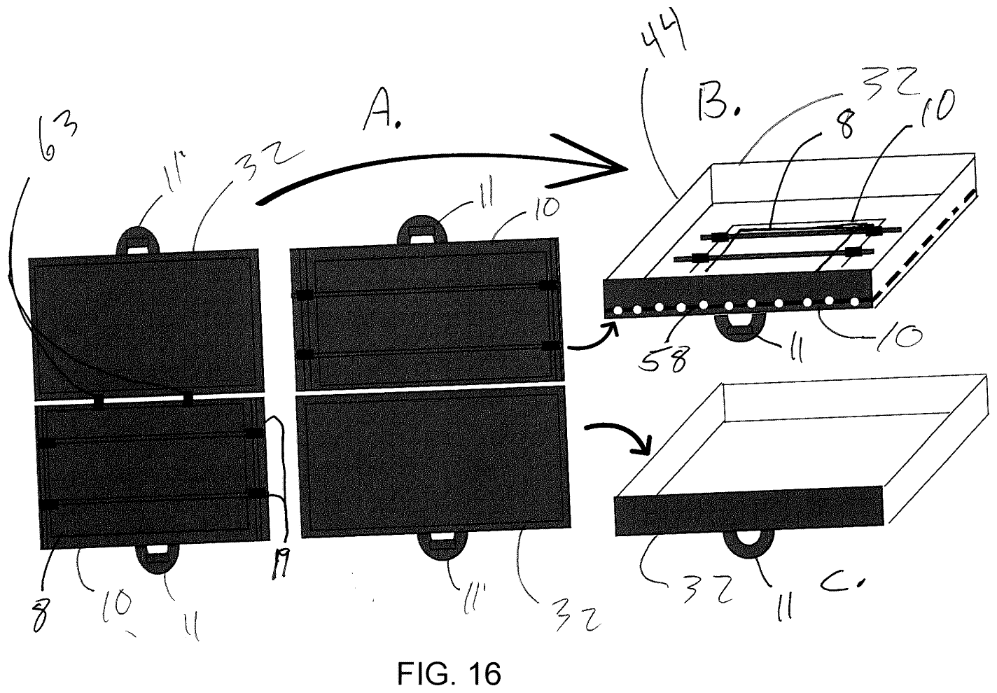

FIG. 17 is a schematic view illustrating applications of the hand support device according to the above preferred embodiment of the present invention.

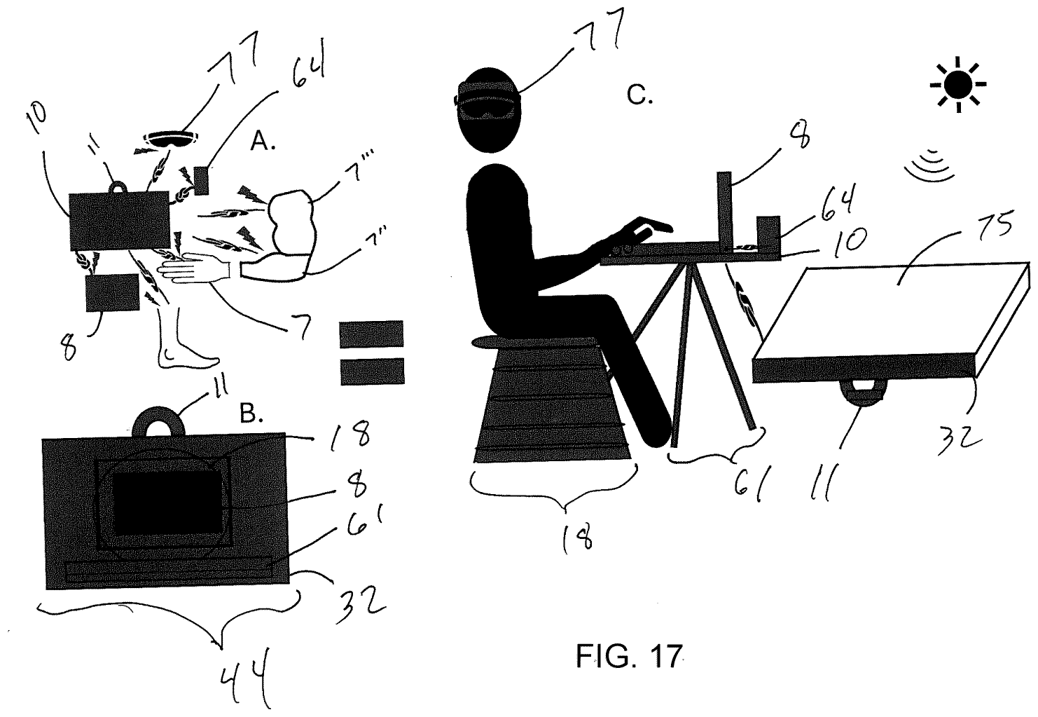

FIG. 18 is a schematic view illustrating other applications of the hand support device according to the above preferred embodiment of the present invention.

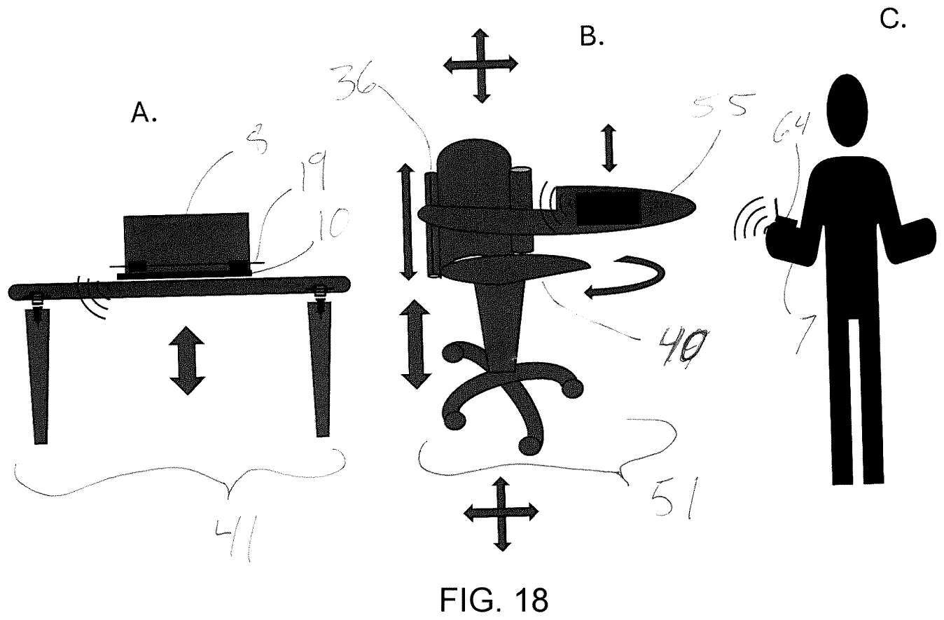

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

In the description of the present invention, unless explicitly stated otherwise and qualified, terms such as “connected,” “attached,” and “fixed” should be construed broadly. For instance, these terms may indicate a permanent connection or a detachable one, or they may refer to a whole unit. They can signify a mechanical linkage, an electrical connection, direct coupling, or indirect interaction through an intermediary medium. Whether these terms imply an internal connection between two elements or an interactive relationship between them will depend on the specific context and the understanding of those skilled in the art.

Throughout this invention, unless explicitly stated otherwise and qualified, when the first feature is described as being “above” or “below” the second feature, this may entail direct physical contact between the two features. Alternatively, it may signify that the first and second features are not in direct contact but are linked through the involvement of additional features. Additionally, the description of the first feature being “above,” “over,” or “on top of” the second feature includes scenarios where the first feature is positioned directly above or diagonally above the second feature or simply means that the first feature is situated at a higher horizontal level than the second feature. Conversely, when the first feature is referred to as “below,” “under,” or “beneath” the second feature, it encompasses cases where the first feature is directly below or diagonally below the second feature or simply implies that the first feature's horizontal height is less than that of the second feature.

In this embodiment's description, terms such as “up,” “down,” “right,” and “left” are used to describe orientations or positional relationships. These descriptions are based on the orientations or positions depicted in the drawings and are employed for ease of explanation and simplification of operation. They should not be construed as indications or implications that the device or element being discussed must possess a specific orientation, be constructed in a particular manner, or operate exclusively in a certain orientation. Furthermore, terms such as “first” and “second” are employed solely for the purpose of distinction in the description and do not carry any particular significance.

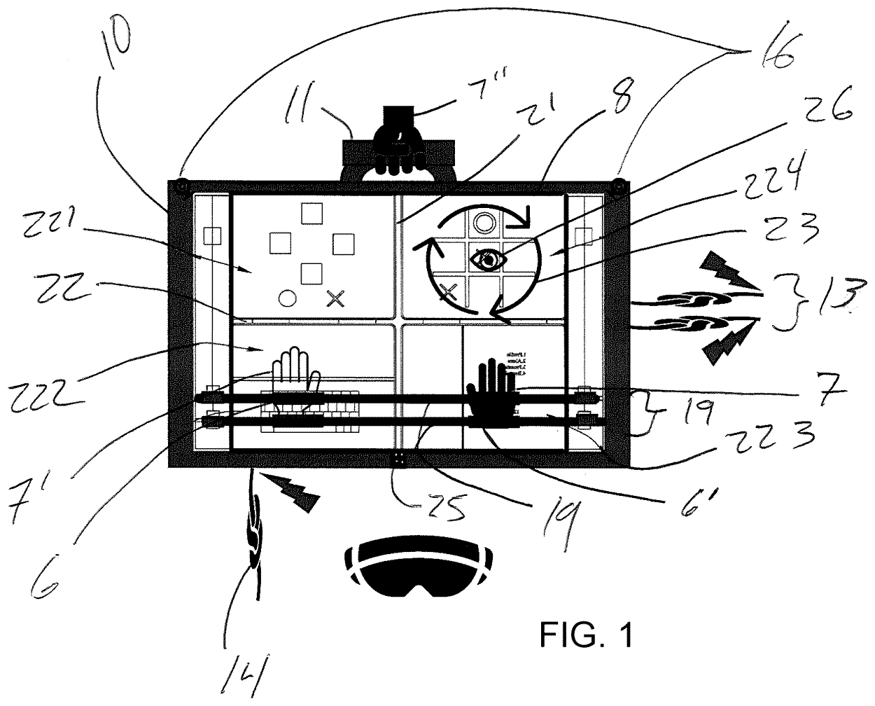

Referring to FIG. 1 of the drawings, a hand support device 10 for input apparatus such as typing apparatus like keyboard, notebook, laptop, or the like according to a preferred embodiment of the present invention is illustrated, wherein the hand support device 10 can be mounted to a keyboard to provide assistance with work like typing and repetitive fingers or wrist movements, and other activities of hands or wrists such as playing games and recreational repetitive activities, while providing various controls performed or imagined, including VR/AR/XR/MR, smart glasses or goggles glasses headset 77. Preferably, a touchscreen electronic computer device 8 and the smart glasses/goggle VR, AR, MR, XR (AI) program display medium headset 77 are interface. The hand support device 10 has various accessories like attachable or built-in cameras 16 for gesture recognition, and one or more bar hand support 19 location, etc. The hand support device is electronically charged wirely or wireless 14 and is also able to electronically charge other electronic devices wirely or wireless 13. The hand support device may or may not have a hand support device handle 11 for portability and to provide angle inclination to the touchscreen electronic device 8, including the entire hand support device 10. Additionally, the hand support device may or may not have a built-in microphone 25 for voice commands.

Alternatively, the invention teaches a hand support device 10 with one or more electronic computers 8 attachable or built-in within the hand support device 10. The electronic computer devices 8 may or may not operate with horizontal sidelines 22, vertical sidelines 21 and or circular sidelines 23 computer functions to separate multiple display programs 221, 222, 223, and 224. The user biological hand 7′ may operate display program 222, while the bionic limb, prosthetic, osseointegration limbs implants, orthotic, exoskeleton electronic device the non-biological limb 7 operates display program 223. It is important to mention that the display programs 221, 222, 223 sand 224, are all separate by the sidelines (21, 22 and or 23) configuration and all the display programs may or may not independently operate at the same time or may be inputting systems for operations of one or more display programs 221, 222, 223, and 224. It is important to mention that the invention teaches a dormant interface 89 for the bionic limbs, prosthetics, osseointegration limbs implants and orthotics, including exoskeletons systems, that will later be further explained. The invention teaches one bionic limb, prosthetic, osseointegration limbs implants, orthotic, or exoskeleton like the bionic hand that is a non-biological limb 7 as an example, it is not limited to one single extremity or limb, like the arm, shoulder or leg. The invention may or may not apply to one or more bionic limbs, prosthetics, osseointegration limbs implants and orthotics, including exoskeletons systems.

At the same time the user may or may not be operating and navigating one or more inputting systems like voice command, eye motion control, retinal recognition, fovea recognition cursor 55 and gesture recognition from the cameras in the smart glasses/goggle VR, AR, MR, XR (AI) program display medium headset 77. For example, the smart glasses/goggle VR, AR, MR, XR (AI) program display medium headset 77 eye motion control, retinal recognition, fovea recognition cursor 55 is controlling the display program 224, as illustrated in FIG. 1. It is important to mention that the multiple display programs 221, 222, 223, 224, the vertical sideline 221, the horizontal sideline 222, the circular program 223, along with the smart glasses/goggle VR, AR, MR, XR (AI) program display medium headset 77 eye motion control, retinal recognition, fovea recognition cursor 55, the touch screen electronic computer 8 and the position of the one or more bar hand support 19, with the biological hand 7′ and with the non-biological limb 7 are all being displayed within the smart glasses/goggle VR, AR, MR, XR (AI) program display medium headset 77. Alternatively, all or partial customized displays may be shared or customized for double display between the physical display touch screen electronic computer 8 and the smart glasses/goggle VR, AR, MR, XR (AI) program display medium headset 77. It is important to mention that the hand support device 10 may or may not have a built-in display touch screen electronic computer 8 or may have a detachable and attached display touch screen electronic computer 8 that produces a portable or fixed smart hand support device 10.

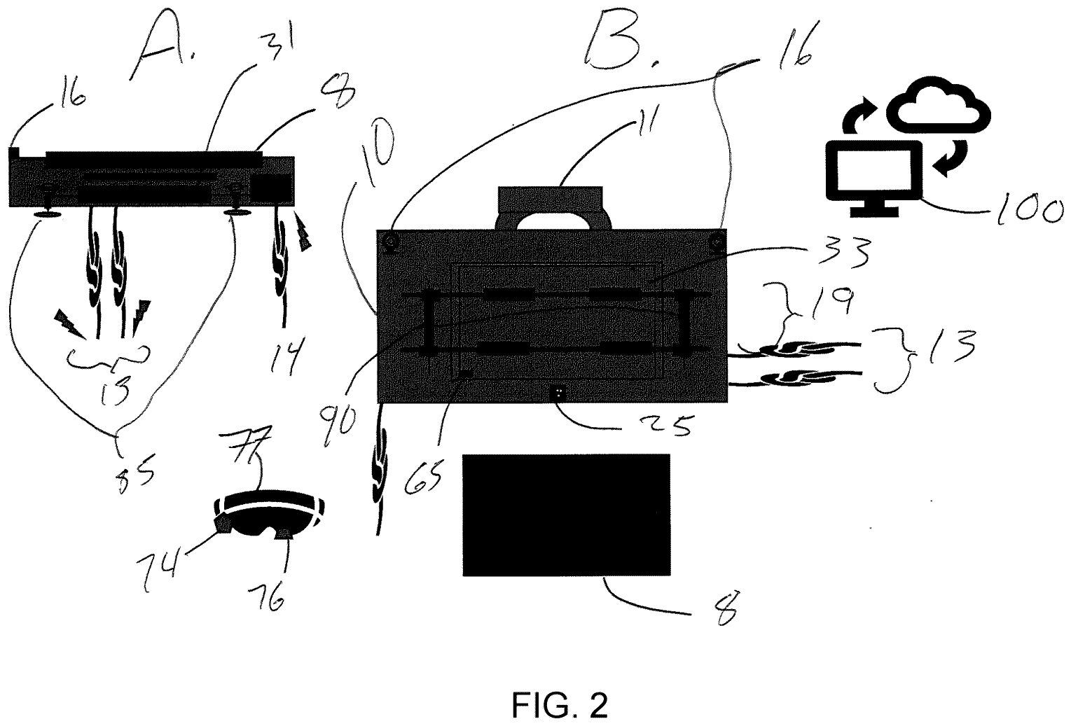

Referring to FIG. 2 of the drawings, a hand support device 10 for input apparatus such as typing apparatus like keyboard, notebook, laptop, tablet or the like according to a preferred embodiment of the present invention illustrates the hand support device 10 interfaces with other separated computer electronic devices 8 with or without a built-in touch screen. It is important to mention that portion A of FIG. 2 illustrates the hand support device 10 with a built-in computer electronic device 8 that is operated with a touch screen system. Alternatively, the hand support device 10 may have a touch screen system that interfaces with other computer electronic devices 8 for controlling and operating any other electronic computer devices 88. Furthermore, the cross view illustrates the hand support device 10 with a built-in charging battery 31 capable of charging other electronic devices irrespective of having a built-in computer electronic device 8. The built in charging battery 31 may also operate the mother board for all the accessories within the hand support device 10, for example the built-in or attachable cameras 16, built-in or attachable speaker/microphone 25, or the built-in or attachable electronic height adjustor 85.

Referring to FIG. 2, portion B illustrates the hand support device 10 as an independent inputting system with interoperability with other computer electronic devices 8 for customization of inputting systems. For example, the hand support device 10 may function without a built-in computer electronic device 8 or a built-in touch screen surface. Alternatively, the microphone/speaker 25 may function the voice command program within the hand support device 10 or may have interoperability within the microphone/speaker of the headset 77 or interoperability within the other microphone/speaker systems of any other computing device 88 that are portable or fix. Furthermore, the hand support device 10 along with the bar hand support 19 system and the cameras 16 system in conjunction with a gesture recognition program may be operated and navigated the computer electronic device 8, any other computing device 88, including the smart glasses/goggle VR, AR, MR, XR (AI) program display medium headset 77. Furthermore, the invention teaches interoperability between the microphone/speaker 25 within the hand support device 10, the camera system 76 and the microphone/speaker 74 within the smart glasses/goggle VR, AR, MR, XR (AI) program display medium headset 77, the camera system 16 within the hand support device 10 all operating at the same time for the purpose of multitasking with or without bionic limbs, prosthetics, osseointegration limbs implants and orthotics, including exoskeletons. It is important to mention that the EMG, EEG and or EcoG signal acquisition (AI) programs may or may not operate one or more functions of the hand support device 10.

Additionally, FIG. 2B illustrates the hand support device 10 accessories, like the attachable hand grip 11 that also function for inclining the hand support device 10 with or without a computer electronic device 8. The hand support device 10 also has a wirely or wireless interface attachment 65 for interfacing with any other computing device 88, the smart glasses/goggle VR, AR, MR, XR (AI) program display medium headset 77 and or the computer electronic device 8. The wirely or wireless interface attachments can be anywhere in the hand support device as illustrated in portions A and B of FIG. 2, wherein the hand support device 10 is illustrated in portion B of FIG. 2 to have a built-in cavity/support attachment system 33 for positioning various size computer electronic devices 8. It is important to mention the one or more bar hand support 19 has accessory sleeves 6 for additional ergonomic and also the accessory sleeves 6 have one or more interface sensors 66 in one or more accessory sleeves 6. Additionally, the sleeves 6 along with the bar support adjuster 90 and the cavity/support attachment system 33. The bar pillars 90 and the one or more bar hand support 19 are adjustable, slide and lock on each side at selected positions to fasten/secure the computer electronic device 8.

When one or more hands with EMG, EEG and or EcoG signal acquisition (AI) programs, or with one or more bionic limbs, prosthetics, osseointegration limbs implants and orthotics, including exoskeletons activates one or more interface sensors 66 that may or may not allow for operation and navigation within the smart glasses/goggle VR, AR, MR, XR (AI) program display medium headset 77, within the computer electronic device 8 or within any other computers 88 at the same time or independently. Alternatively, after interface sensors 66 and when one or more hands with EMG, EEG and or EcoG signal acquisition (AI) programs, or with one or more bionic limbs, prosthetics, osseointegration limbs implants and orthotics, including exoskeletons are off may or may not activated the voice command program in the server cloud 100 or within the hand support device 10, where the user inputting information are acquired by the microphone/speaker 25 to operated and navigate the smart glasses/goggle VR, AR, MR, XR (AI) program display medium headset 77, the computer electronic device 8, and or any other computer 88, at the same time or independently. Additionally, when either hand is off, any interface sensors 66 that may or may not active the gesture recognition/sign language recognition program in the server cloud 100 or in the hand support device 10. The hand support device cameras 16 and microphone/speaker 25 may also be assisted by the smart glasses/goggle VR, AR, MR, XR (AI) program display medium headset 77 own camera 76 and microphone/speaker 74. The invention teaches interchangeability and interoperability among inputting systems, without limitations, within one or more built-in processor and or server cloud 100 processors.

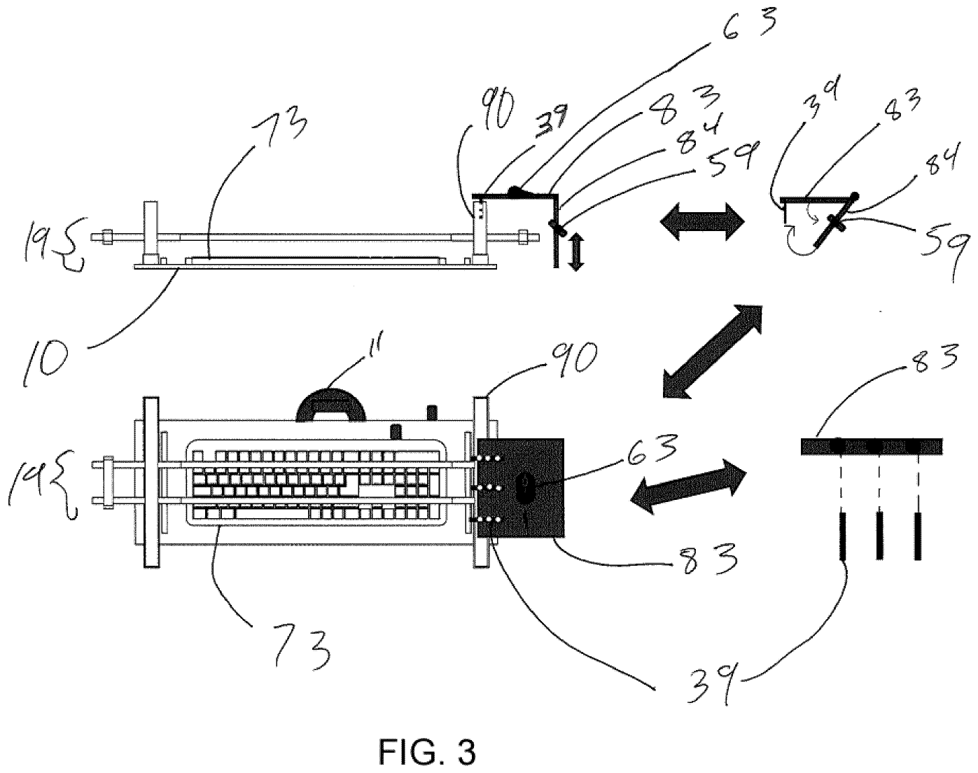

Referring to FIG. 3, illustrates the hand support device 10 able to interface and interlink while electronically re-charging wirely or wireless, any computer electronic device 88, including a touch screen display keyboard 73, physical e-mouse 63 (cursor control), etc. Furthermore, the hand support device 10 has an e-mouse accessories platform 83 that is be foldable or collapsible and has universal attachments 49 or locking rods 39 horizontal or vertically for securing the e-mouse accessories platform 83 with the hand support device 10, while operating with biologic hand or with one or more bionic limbs, prosthetics, osseointegration limbs implants and orthotics, including exoskeletons. The e-mouse accessory platform 83 is supported by the foldable/collapsible leg platform 84, additionally the leg adjuster 59 provides various heights for the e-mouse accessory platform 83. The USB connection 31 can be for wireless connection or for wirely connection, within any electronic device including the hand support device 10. It is important to mention that the hand support device 10 wirely or wireless interfaces with any other computer device 88, the physical e-mouse 63 and smart glasses/goggle VR, AR, MR, XR (AI) program display medium headset 77, with or without wirely or wireless EMG, EEG and or EcoG signal acquisition (AI) programs.

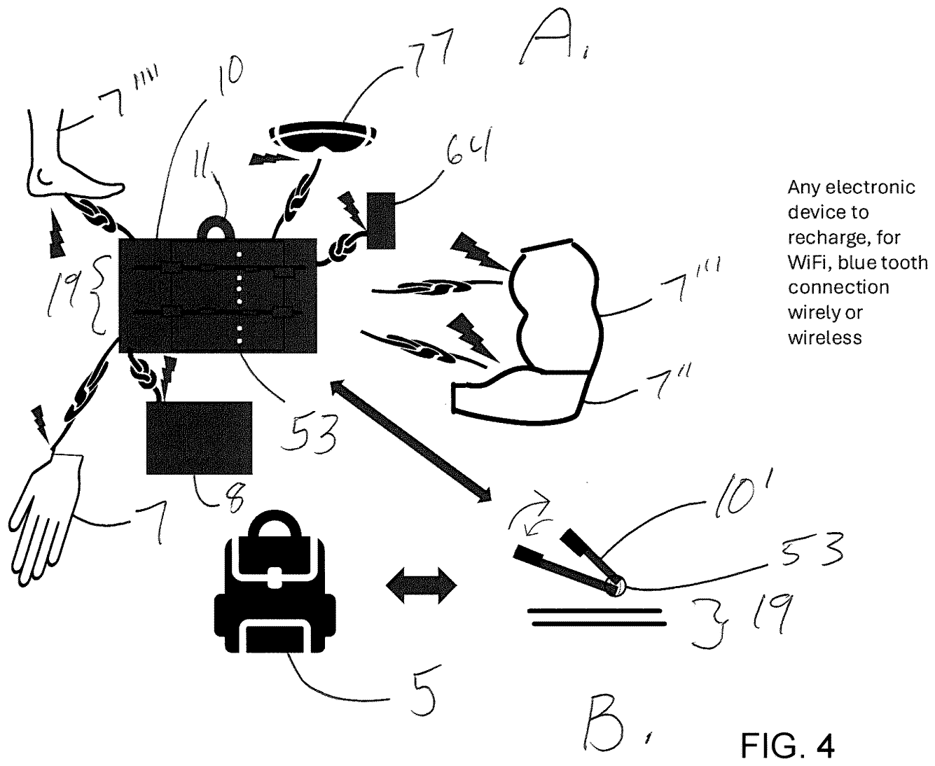

Referring to FIG. 4A, illustrates the hand support device 10 able to interface and interlink while electronically re-charging wirely or wireless, any electronic device, like bionic limbs, prosthetics, osseointegration limbs implants and orthotics, including exoskeletons. It is important to mention that through the hand support device 10 the user is able to interface with any computer electronic device 8, smart glasses/goggle VR, AR, MR, XR (AI) program display medium headset 77, smartphone 64, non biological limb 7. The interface interlink can be any type WIFI, blue tooth, wireless or wirely (USB) connection, etc. In other words, the user may type and or text within the hand support device 10 when operating the smartphone 64, computer electronic device 8, smart glasses/goggle VR, AR, MR, XR (AI) program display medium headset 77. Furthermore, the hand support device 10 may access any computer application (app) for any computer electronic device 8 or other electronic devices 88 with interoperable software capacity. It is important to mention that the hand support device 10 has folding hinges 53. The foldable/collapsible hand support device 10′ is possible by removing the one or more support bars 19 as illustrated in portion B of FIG. 4. Furthermore, the foldable/collapsible hand support device 10′ way folded or collapse at the folding hinges 53 for transporting the hand support device 10′ within a gym bag, carryon, or any other housing container 5 and is able to continue electrically re-charge other electronic device, as mentioned earlier within any housing container of the foldable/collapsible hand support device 10', including the non-foldable/collapsible hand support device 10.

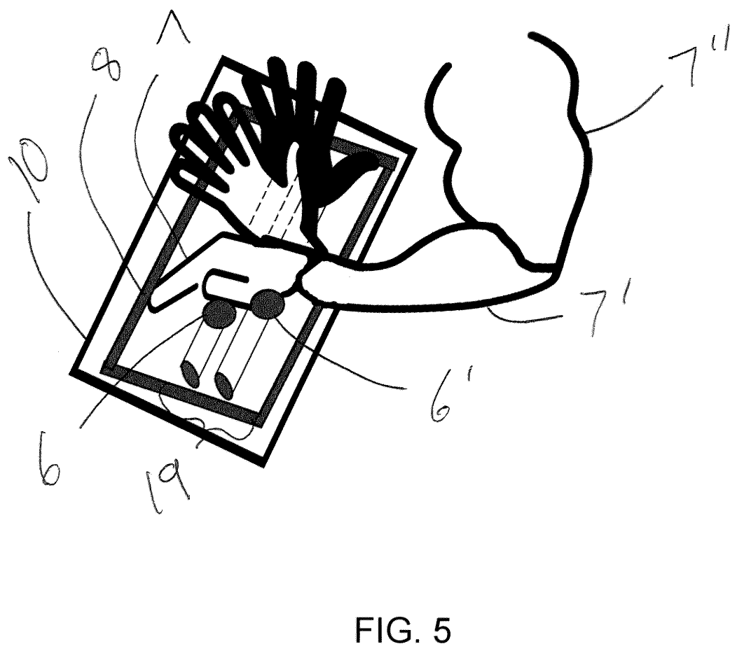

Referring to FIG. 5, the hand support device 10 is able to interface and interlink while electronically re-charging wirely or wireless, any electronic device, like bionic limbs, prosthetics, osseointegration limbs implants and orthotics, including exoskeletons. It is important to mention that the hand support device 10 allows the EMG skin sensors, EEG nerve scalp implants, EcoG brain implants and or EMG myocyte implants to operated individually or collectively. In other words, the hand support device 10 provides the upper extremity neuromuscle relaxation that enables selective isolation of signal acquisition action to produce the correct EMG, EEG and or EcoG signal acquisition (AI) programs, including brain signal for the corresponding inputting command selected by the user. Furthermore, the hand support device 10 enables relaxation of bilateral upper extremities to produce the proper signal acquisition from either alternating upper extremity. Additionally, the hand support device 10 provides relaxation that allows scalability for multiple sequential signal acquisitions from the same upper extremity or from both upper extremities independently and at the same time. It is important to mention that the invention teaches imagined, performed or partially performed inputting commands through the EMG, EEG and or EcoG signal acquisition (AI) programs from one or more biological hand 7′ operating with a wirely or wireless sensors 80, within the EMG wrist band 40, within the sensors EMG hand glove 41, or within the sensors EMG arm sleeve 39. It is important to mention FIG. 5 alternatively, illustrates one or both non-biological limbs 7 being supported on the hand support device 10 with one or more bars 19 and activating one or more sleeve sensor interface 66 for activating the Brain-computer interface (BCI) and or the Brain machine interface (BMI). Preferably, both the Brain-computer interface and or the Brain machine interface (BCI/BMI) operate through the EMG, EEG and or EcoG signal acquisition (AI) program in the hand support device 10. It is important to mention that the signal acquisition (AI) programs for (BCI/BMI) technology can come from any other computing device 88, like the Meta-Ray-Ban Smart Glasses system.

In one or more biological hands 7′ or in one or more non-biological limbs 7 the user can have utilization of non-invasive external electrodes on the skin EMG or external electrodes on the scalp EEG. The one or more non-biological limbs 7 may or may not have additional sensor EMG wearables like the biological hands 7′, like the sensors EMG wrist band 40, sensors EMG hand glove 41, or sensors EMG arm sleeve 39 because the hand support devices 10 may or may not convert the EMG signal acquisition sensors that are built for controlling the movement of the bionic limbs, prosthetics, osseointegration limbs implants, orthotics, and exoskeletons system wearables and switches them to interface with other any other computer 88, without or with limited movements within the bionic limbs, prosthetics, osseointegration limbs implants, orthotics, and exoskeletons system wearables.

It is important to mention that while one or both upper extremities are being supported by the hand support device 10, one or both bionic limbs, prosthetics, osseointegration limbs implants and orthotics, including exoskeletons may or may not be electrical re-charge. It is important not to overlook the posture alignment benefits that the hand support device 10 provides to the bilateral shoulders, cervical spine, thoracic spine and lumbar spine with or without bionic limbs, prosthetics, osseointegration limbs implants and orthotics, including exoskeletons system wearables. Additionally, when one or more bionic limbs, prosthetics, osseointegration limbs implants, orthotics, and exoskeleton system wearable or when one or more biological hands 7′ are position on the hand support device 10, for operating the (BCI/BMI) within the hand support device 10, it may or may not operate with wirely or wireless semi-invasive electrodes EEG sensors or ECoG sensors. Furthermore, the hand support device 10 may or may not operate with wirely or wireless invasive implant electrodes ECoG that may or may not be provided to operate the bionic limbs, prosthetics, osseointegration limbs implants, orthotics, and exoskeleton wearable system.

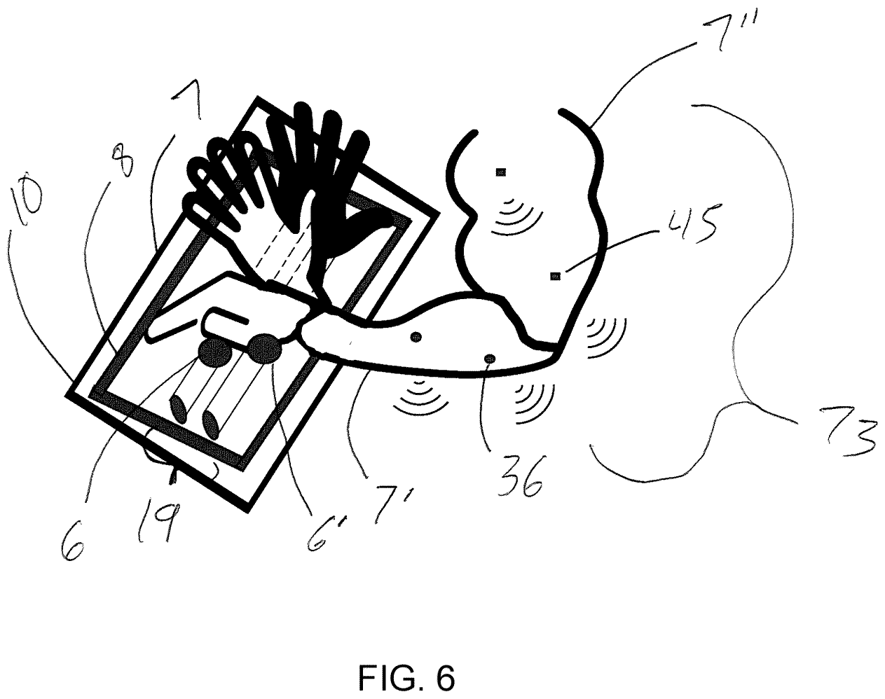

Referring to FIG. 6, the hand support device 10 is able to interface and interlink with bionic limbs, prosthetics, osseointegration limbs implants and orthotics, including exoskeletons while electronically re-charging wirely or wireless in the anatomical ergonomic neutral position 73. The anatomical ergonomic neutral position 73 enables the EMG, EEG and or EcoG signal acquisition (AI) programs to have scalability with the most precision for multitasking. The EMG, EEG and or EcoG signal acquisition (AI) program 36 and myocyte sensor implant 45 can work independently or in combination for scalability and robust performance. Both signal acquisitions systems are primarily used to advance the operational movements of the bionic limbs, prosthetics, osseointegration limbs implants, orthotics, and exoskeletons system wearables. Through the hand support device 10 with (BCI/BMI) technology or any other similar technology any biological signal acquisition can be used to interface with any other computing device 88, including the attachable detachable touch screen 8. Preferably, the hand support device 10 is used in a control environment to optimize productivity, reduce anatomic pathological stressors physically and mentally, as the means to increase wellness. It is important to mention that operating any AR, MR, XR (AI) program display medium headset 77 or any other electronic device 88 through the hand support device 10 software or computer application increases the performance. In other words, with or without bionic limbs, prosthetics, osseointegration limbs implants and orthotics, including exoskeletons wearable system, the internal signal acquisition system, the user is in full operational with interoperability functioning within other inputting systems for the purpose of operating and navigating multiple computing systems. Additionally, any computing function may be customized through the cloud servers.

Ideally the hand support device has the artificial intelligence center (BCI/BMI) technology for operating VR, AR, MR, XR devices smart glasses/goggles with artificial intelligence programs and navigating programs through EMG, EEG and or EcoG signal acquisition (AI) programs. For example, in the last two embodiments the user controls the VR, AR, MR, XR devices smart glasses/goggles artificial intelligence (AI) program independently and in conjunction with the EMG, EEG and or EcoG signal acquisition (AI) programs. The hand support device enables the EMG, EEG and or EcoG signal acquisition (AI) programs to have precision signal acquisition for scalability. In other words, the hand support device provides physical support of the upper extremity, cervical spine, thoracic spine and lumbar spine enabling the (AI) programs to have a neutral baseline biological signal acquisition recording (imagined, performed or partially performed) for the selected function. The hand support eliminates unwanted movements, motion artifacts, crosstalk artifacts, Physiological artifacts, Ocular artifacts, and provides proper electrode placement. The hand support device has advance filtering system for motion artifact reduction, environmental power line interference filters system, etc. the hand support device can helps to record and support mild cyclic tremors to enable proper interpretation of EMG, EEG and or EcoG signal acquisition (AI) programs, when pre-programing the EMG, EEG and or EcoG signal acquisition (AI) programs to the selected executive function.

It is important to mention that the smart glasses/goggle VR, AR, MR, XR (AI) program display medium have video cameras recording capacity. The user can record audio and video within the VR, AR, MR, XR devices smart glasses/goggles to program with artificial intelligence pre-program functions. Therefore, through the hand support device the user is able to program and record scalable EMG, EEG and or EcoG signal acquisition (AI) programs in a control environment with advance precision for pre-programing the smart glasses/goggle VR, AR, MR, XR (AI) program devices. In other words, the smart glasses/goggle VR, AR, MR, XR (AI) program display medium recordings can be displayed at a later time on demand for scalable pre-programing of the EMG, EEG and or EcoG signal acquisition (AI) programs with any smart glasses/goggle VR, AR, MR, XR (AI) program devices.

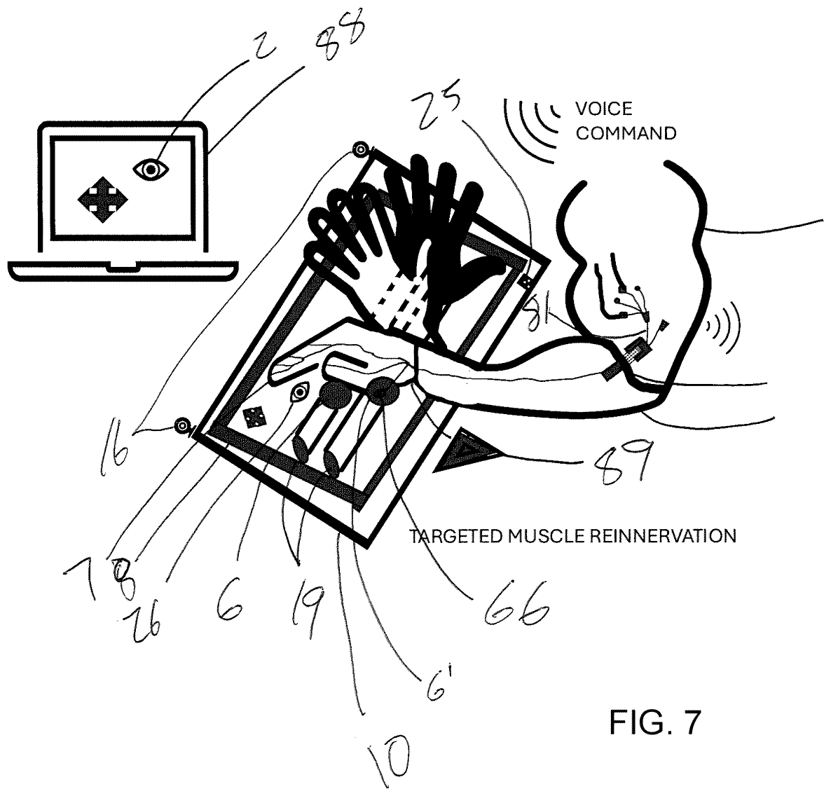

Referring to FIG. 7, the interoperability of the hand support device 10 is embodied as an independent inputting system and as a collective inputting system with any other computing device 88, with the smart glasses/goggle VR, AR, MR, XR (AI) program display medium headset 77, with the computer electronic device 8 and within (BCI/BMI) technology operational digital systems. The invention teaches targeted muscle reinnervation 81 (TMR), osseointegration implantation, free muscle transplant, biocompatible wire or connective material and electrodes for signal amplification operational within the bionic limbs, prosthetics, osseointegration limbs implants and orthotics, including exoskeletons systems, in non-biological limb 7 systems that is supported by the hand support device 10. Targeted muscle reinnervation 81 (TMR) is a surgical procedure that helps with the mechanical movements operations of the bionic limbs, prosthetics, osseointegration limbs implants and orthotics, including exoskeletons systems and secondary also helps alleviate neuromas and phantom limb syndrome. Currently targeted muscle reinnervation 81 (TMR) is also being performed with free muscle transplantation and implantation of electrodes 45 for signal amplification to operates and control movement of the bionic limbs, prosthetics, osseointegration limbs implants and orthotics, and is also possible to including exoskeletons systems.

Targeted muscle reinnervation (TMR) 81 with or without osseointegration surgery, allows for amplification and signal acquisition so that the bionic limbs, prosthetics, osseointegration limbs implants and orthotics, including exoskeletons systems produce a more robust physical outcome. For example, when controlling a touch screen device computer, we physically want to extend our biological finger or the non-biological finger to produce the physical contact in the touch screen for the final outcome within the computer to generate a digital response. Through the hand support device 10 the bionic limbs, prosthetics, with or without osseointegration limbs implants and orthotics, including exoskeletons systems are dormant or partially move (customized) from any or all physical movement, in the dormant interface 89, the software are interlinked wirely or wireless to any smart computing electronic device 88, smart glasses/goggle VR, AR, MR, XR (AI) program display medium headset 77, and or computer electronic device 8. The invention teaches the preservation of the bi-directional communication between the biological neuromuscular signal and the bionic limbs, prosthetics, osseointegration limbs implants and orthotics, including exoskeletons systems without unnecessary mechanical movements during computer operations within the hand support device 10 systems, as the bionic limbs, prosthetics, osseointegration limbs implants and orthotics, including exoskeletons are also electrically re-charging. The Bi-directional communication targeted muscle reinnervation continues to be powered up for signal acquisition during computer digital work. Thereby, additional energy can be re-directed away from the bionic limbs, prosthetics, osseointegration limbs implants and orthotics, including exoskeletons mechanical movements for a faster charging of the bionic limbs, prosthetics, osseointegration limbs implants and orthotics, including exoskeletons systems during the dormant state as the movements are reduced or completely stopped. In dormant interface 89, the bionic limbs, prosthetics, osseointegration limbs implants and orthotics, including exoskeletons systems can be electrically re-charged by the hand support device 10, as earlier mentioned and the signal acquisition continues to be fully operational for computer digital work. It is important to mention the hand support device 10 is also interface, interoperability, wirely or wireless linked to any smart computing electronic device 88, to a computer electronic device 8, and or smart glasses/goggle VR, AR, MR, XR (AI) program display medium headset 77, allowing for the bi-directional communication for signal acquisition to generate digital response within the internet, intranet, without a physical inputting medium or with partial physical medium from the one or more non biological limbs 7.

For teaching purposes, the touch screen of the computer electronic device 8 is divided by the vertical sideline 21, where the touch screen portion 223 is controlled the non-biological limb 7 side and is not operation to tactile or partially operational to selected gestures within the touch screen device 8 or for the gesture program from the cameras 16 within the hand support device 10. At the same time in the touch screen of the biological hand 7′ portion 222, all the touch screen components are fully operational in relation to the non-biological hand 7. The vertical sideline separates the nonfunctional touch screen portion from the touch screen operational area for the biological hand 7′, as illustrated in FIG. 1. The invention teaches the target muscle reinnervation (TNR) 81 for inputting through a non-physical medium with or without bionic limbs, prosthetics, osseointegration limbs implants and orthotics, including exoskeletons systems with reduction or complete elimination of physically robotic motion to operate the touch screen in the computer electronic device 8 with the utilization of the sideline program within the computer electronic device 8 in the hand support device 10. The vertical sideline 21 separates the nonfunctional touch screen, while allowing the biological hand 7′ to continue full utilization of the touch screen in the computer electronic device 8 of the hand support device 10, as illustrated in FIG. 1. Furthermore, in the portion 223 of the touch screen that is separated by the vertical sideline 21 with the non-biological limbs 7, prosthetics, osseointegration limbs implants and orthotics, including exoskeletons systems become operational by the signal acquisition (EMG), voice command, gesture and or the smart glasses/goggle VR, AR, MR, XR (AI) program display medium headset 77 (EEG/EcoG) non-tactile medium and or non-physical medium, including gesture performed or imagined, eye retinal, line of sight, voice commands and or sign language program capture by the cameras 76 in the smart glasses/goggle VR, AR, MR, XR (AI) program display medium headset 77, capture by the cameras 16 in the hand support device 10 or by the voice command microphone/speaker 25 in the hand support device or in the microphone/speaker 74 in the smart glasses/goggle VR, AR, MR, XR (AI) program display medium headset 77. It is important to mention that the CPU in the bionic limbs, prosthetics, with or without osseointegration limbs implants and orthotics, including exoskeletons systems interfaces with other CPUs systems from any smart computing electronic device 88.

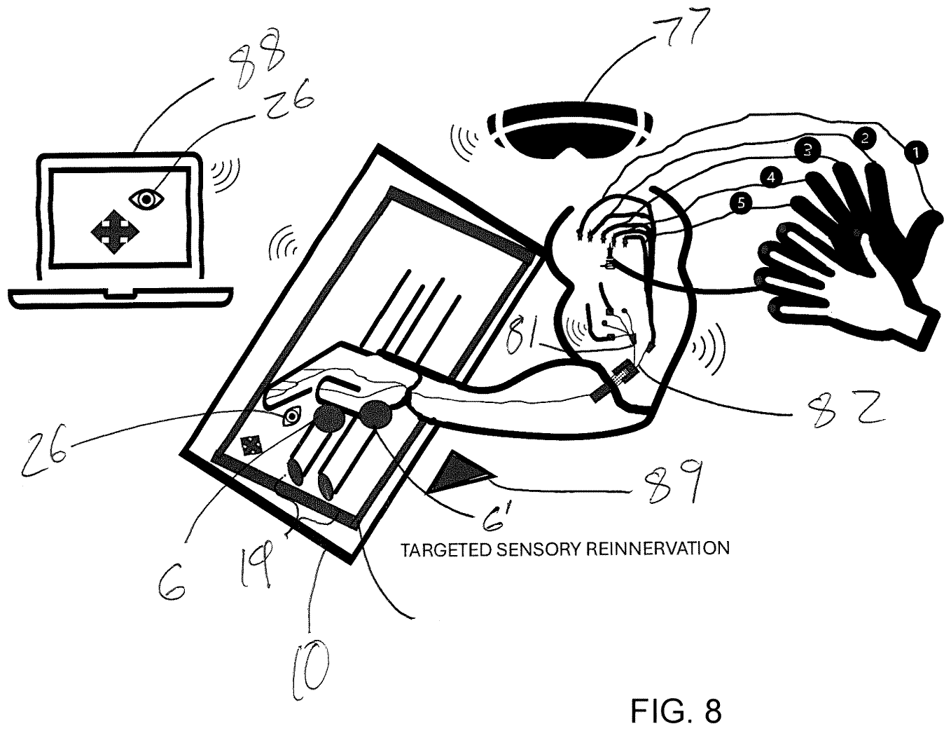

Referring to FIG. 8, the interoperability of the hand support device 10 is embodied as an independent inputting system and as a collective inputting system with any smart computing electronic device 88, with the smart glasses/goggle VR, AR, MR, XR (AI) program display medium headset 77, with one or more computer electronic device 8 and within AI/BCI/BMI operational digital systems. The targeted sensory reinnervation (TSR) 82 surgical operation is performed to provide bionic limbs, prosthetics, osseointegration limbs implants and orthotics, including exoskeletons systems another path of sensory. In other word, the user of bionic limbs, prosthetics, osseointegration limbs implants and orthotics, including exoskeletons systems have built-in sensors at pre-selected areas for providing a feedback sensory to the user at preselected location in the user's skin, as the means for touch through the usage of bionic limbs, prosthetics, osseointegration limbs implants and orthotics, including exoskeletons systems, describe as the “primary sensory”.

The invention teaches a “secondary sensory”, wherein users may or may not feel the primary sensory as originally intended from the bionic limbs, prosthetics, osseointegration limbs implants and orthotics, including exoskeletons system where the distal sensory electrodes do not deliver the impulse with physical touch of the bionic limbs, prosthetics, osseointegration limbs implants and orthotics, including exoskeletons system sensors electrodes independently or together with targeted muscle reinnervation (TMR) 81. In other words, when the bionic limbs, prosthetics, osseointegration limbs implants and orthotics, including exoskeletons system, are position over the hand support device 10 for computer operations the “secondary sensory” path is activated in relation to the bionic limbs, prosthetics, osseointegration limbs implants and orthotics, including exoskeletons system having a reduction or a completely stopped of sensory impulses from the touch sensors within the bionic limbs, prosthetics, osseointegration limbs implants and orthotics, including exoskeletons systems. The invention teaches the preservation of the bi-directional communication of the targeted sensory reinnervation (TSR) 82. Additionally, the skin digital sensors 12 that consist of 5 separated skin regions that mimic each of the 5 fingers for sensory that can be stimulated at the same time or corresponding to contact of an object in relation to the non-biological 7 (5) fingers of the bionic limbs, prosthetics, osseointegration limbs implants and orthotics, including exoskeletons systems to provide sensory, with or without additional haptic/sensory from the bionic limbs, prosthetics, osseointegration limbs implants and orthotics, including exoskeletons systems during computer operations within a hand support device 10 during the dormant interface 89 and while operating any smart computing electronic device 88, smart glasses/goggle VR, AR, MR, XR (AI) program display medium headset 77, one or more computer electronic device 8 wherein the bionic limbs, prosthetics, osseointegration limbs implants and orthotics, including exoskeletons systems have partial or complete stoppage of sensory in relation to the “primary sensory”. In the “secondary sensory” the bionic limbs, prosthetics, osseointegration limbs implants and orthotics, including exoskeletons systems are interface, integrated, wirely or wireless linked to the hand support device 10, the smart glasses/goggle VR, AR, MR, XR (AI) program display medium headset 77, any smart computing electronic device 88 and or one or more computer electronic device 8, that enable the user to have sensory within the (digital medium) computer operations in the internet, intranet non-physical medium, not as intended in the “primary sensory”. In other words, within the dormant interface 89 and during interface with the smart glasses/goggle VR, AR, MR, XR (AI) program display medium headset 77 line of sight fovea cursor 26 program the sensory impulses orientation is changed, not originating within the touch sensors of the bionic limbs, prosthetics, osseointegration limbs implants and orthotics, including exoskeletons system. Alternatively, the targeted sensory innervation (TSI) 82 is operational and is being provided sensory impulses from the smart glasses/goggle VR, AR, MR, XR (AI) program display medium headset 77 line of sight cursor 26 for direction, movement and touch as the smart glasses/goggle VR, AR, MR, XR (AI) program display medium headset 77 line of sight fovea cursor 26 is moving during computer operations with or without additional impulses from the touch sensors as originally intended in the bionic limbs, prosthetics, osseointegration limbs implants and orthotics, including exoskeletons system. It is important to mention the “secondary sensory” mention earlier for the targeted sensory innervation 82 can also be reproduced with interoperability between any smart computing electronic device 88, the smart glasses/goggle VR, AR, MR, XR (AI) program display medium headset 77, the hand support device 10 with or without the computer electronic device 8.

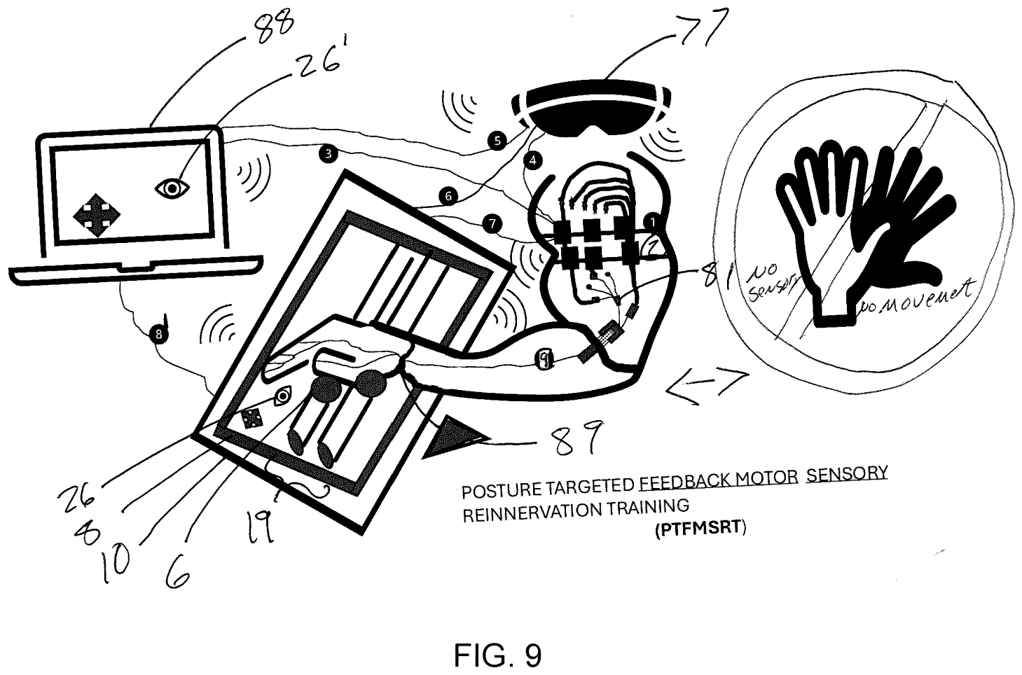

Referring to FIG. 9, the interoperability of inputting systems with the hand support device 10 is embodied as an independent inputting system and as a collection of inputting systems with any smart computing electronic device 88, smart glasses/goggle VR, AR, MR, XR (AI) program display medium headset 77, with one or more computer electronic device 8 within AI/BCI/BMI operational digital systems, Including targeted muscle reinnervation 81 and targeted sensory reinnervation 82 with or without bionic limbs, prosthetics, osseointegration limbs implants and orthotics, including exoskeletons systems, performed or imagined signal acquisition for independent performance or for collective performance in a sequential time frame or at the same time to produce targeted feedback motor and sensory reinnervation during digital operations with or without bionic limbs, prosthetics, osseointegration limbs implants and orthotics, including exoskeletons systems. Furthermore, FIG. 9 illustrates the scalability that the hand support device 10 provides to computer operations from the target sensory intervention (TSI) 82 with the signal amplifier wearable 1 and from the targeted muscle innervation (TMI) 81 with the signal amplifier wearable 2. For example, through the targeted muscle innervation (TMI) 81 and the targeted sensory innervation 82 the user can operate the smart glasses/goggle VR, AR, MR, XR (AI) program display medium headset 77, retinal or fovea line of sight cursor 26 program, in this example the user produces an imagined, performed, or partially performed gesture through the EMG targeted muscle in innervation 81 for signal acquisition for activating the smart glasses/goggle VR, AR, MR, XR (AI) program display medium headset 77 retinal or fovea line of sight cursor 26 program. Additionally, the skin digital sensors 12 that consist of 5 separated skin regions that mimic each of the 5 finger sensory are stimulated at the same time or corresponding to contact of an object in relation to the 5 finger sensory, when the smart glasses/goggle VR, AR, MR, XR (AI) program display medium headset 77 retinal or fovea, line of sight fovea cursor 26 stopes the e-cursor from moving in any direction then the stimulation is stopped or activated in one or more of the 5 skin digital sensors 12. Alternatively, the stimulation of all 5 digits can initiate impulse sensation like vibration feature which is used to notify the user of the ongoing computer selections and direction of movement within the digital medium, for example movement of the e-cursor within the medium. Alternatively, when the smart glasses/goggle VR, AR, MR, XR (AI) program display medium headset 77 retinal or fovea line of sight program cursor 26 is moving right or left, up or down the one or more of the 5 skin digital sensors 12 are simulating corresponding to the one or more of the 5 skin digital sensors in relation to the direction or operation of the e-cursor within the digital medium. In other words, during the movement of the e-cursor by the targeted muscle innervation (TMI) 81 the targeted sensory innervation (TSI) 82 is also operational mimicking biological motor and sensory at the same time for controlling digital work, like the E-mouse, e-cursor or swiping, etc. The invention teaches that the primary function of the targeted muscle innervation (TMI) 81 and that the primary function of the targeted sensory innervation (TSI) 82 are for operating bionic limbs, prosthetics, osseointegration limbs implants and orthotics, including exoskeletons is interchangeable for operate any electronic computer device 88 with or with motion of the bionic limbs, prosthetics, osseointegration limbs implants and orthotics, including exoskeletons system.

Alternative, in (TSI) 82 one or more skin sensor within the skin digital sensory 12 may be activated for directional movement of the digital e-cursor within the smart glasses/goggle VR, AR, MR, XR (AI) program display medium headset 77 or in any other electronic computer 88, giving the user a directional indication in relation to the direction of the e-cursor, e-mouse, as it is moving. For example, through the targeted sensory innervation (TSI) 82 the skin digital sensory 12 that is associated with the pinky finger is activated, when the smart glasses/goggle VR, AR, MR, XR (AI) program display medium headset 77 retinal program, fovea program, or line of sight program cursor are moved to the right side, or vice versa or any other digital display. It is important to mention the orientation of the skin digital sensory 12 can be customized by the user. Furthermore, once the e-cursor is positioned on the selected digital function the targeted muscle innervation (TMI) 81 is activated to e-click or e-swipe, etc., to generate a response from the targeted muscle innervation signal acquisition with or without sensory from the targeted sensory innervation or vice versa, for the purpose of non-physical motion nor tactile dexterity digital medium operations. Furthermore, the user may or may not performed any physical movement with the bionic limbs, prosthetics, osseointegration limbs implants and orthotics, including exoskeletons systems nor is the user required to have dexterity for the “primary sensory” while operating with any bionic limbs, prosthetics, osseointegration limbs implants and orthotics, including exoskeletons systems, and while the users bilateral upper extremities are position on the hand support device 10. It is important to mention that at the same time the biological hand 7′ is also able to operate the touch screen in the computer electronic device 8 that is built into the hand support device 10 or attached to the hand support device 10, as illustrated in FIG. 1. It is important to mention that the combination of the targeted muscle innervation (TMI) 81 and the targeted sensory innervation (TSI) 82 are scalable for imagined, performed or partially performed EMG, EEG and or EcoG signal acquisition from neuromuscular, sensory region, gesture, touch points, grip, motion, etc. FIG. 7 and FIG. 8 illustrate interoperability with any bionic limbs, prosthetics, osseointegration limbs implants and orthotics, including exoskeletons systems and within any electronic computing device 88, including the hand support device 10 and it does not illustrated any limitations, it teaches multiple sequential functions for operations of digital work that are scalable from the hand support device artificial intelligence programing, in relation to the targeted muscle innervation (TMI) 81 and targeted sensory innervation (TSI) 82 interoperability together the bionic limbs, prosthetics, osseointegration limbs implants and orthotics, including exoskeletons systems with other computer devices 88, smart glasses/goggle VR, AR, MR, XR (AI) program display medium headset 77 and computer electronics device 8 touch screen surfaces, working at the same time or in a timely manner. In other words, FIG. 7-8 mimic physical movement and physical sensory for operating the bionic limbs, prosthetics, osseointegration limbs implants and orthotics, including exoskeletons systems to work in the physical world.

In contrast, FIG. 9 mimics physical movement and physical sensory for inputting within the digital medium of computer operations, without any physical movement and without any physical sensory originating from the bionic limbs, prosthetics, osseointegration limbs implants and orthotics, including exoskeletons systems, when position on the hand support device 10. In other word, the digital movement and the digital sensory are operational through the interface system (wirely or wireless 1-2) with the hand support device 10 together with the smart glasses/goggle VR, AR, MR, XR (AI) program display medium headset 77 (wireless or wirely 6), the hand support device 10 together with one or more amplifier 1 or 2 (wireless or wirely 7), the hand support device 10 together with any smart computing electronic device 88 (wireless or wirely 8), the hand support device 10 together with the bionic limbs, prosthetics, osseointegration limbs implants and orthotics, including exoskeletons systems (wireless or wirely 9). Furthermore, one or more signal acquisition from the one or more amplifiers 1 or 2 interfaces directly with the smart glasses/goggle VR, AR, MR, XR (AI) program display medium headset 77 artificial intelligence (wirely or wireless 4), interfaces with any smart computing electronic device 88 artificial intelligence (wirely or wireless 3). Additionally, the smart glasses/goggle VR, AR, MR, XR (AI) program display medium headset 77 interfaces with any smart computing electronic device 88 (wirely or wireless 5) or any interface through the internet, intranet, quantum computing, super computers, any digital means with independent servers or through the cloud servers 100. It is important to mention all signal acquisition for inputting originate from the same surgeries for operating bionic limbs, prosthetics, osseointegration limbs implants and orthotics, and or exoskeletons systems and or from the same operational systems for controlling the bionic limbs, prosthetics, osseointegration limbs implants and orthotics, and or exoskeletons systems as intended to provide the physical movement and the physical sensory for physical environmental operations.

Additionally, the targeted muscle innervation (TMI) 81 and the targeted sensory innervation (TSI) 82 may or may not require wirely or wireless sensors. The invention teaches human machine interface (HMI) 83 within the hand supposition device 10, where the sensory delivery and the motor delivery within the bionic limbs, prosthetics, osseointegration limbs implants and orthotics, including exoskeletons systems are customized for signal acquisition and operate with any CPU within any smart computing electronic device 88, including the hand support device 10. For example, through the hand support device 10 the bionic limbs, prosthetics, osseointegration limbs implants and orthotics, including exoskeletons systems may or may not move when mimicking a sensory or motion response within the digital medium. In other words, the hand support device 10 provides proper posture alignment for the bilateral upper extremities, cervical spine, thoracic spine, lumbar spine with one or more bionic limbs, prosthetics, osseointegration limbs implants and orthotics, including exoskeletons systems, while it is electrically re-charging any electronic device including the bionic limbs, prosthetics, osseointegration limbs implants and orthotics, and or exoskeletons systems. It is at this point that the interface for the sensory and motor of the bionic limbs, prosthetics, osseointegration limbs implants and orthotics, including exoskeletons systems is stopped or partially functions in the physical world. The invention teaches the sensory and motor operations of the bionic limbs, prosthetics, osseointegration limbs implants and orthotics, including exoskeletons systems are interchangeable for operational within the digital world and in the physical world. In other words, the invention teaches interoperability in realtime interchanging workload between the digital world and the physical world allowing for the hand support device 10, smart glasses/goggle VR, AR, MR, XR (AI) program display medium headset 77, computer electronic device 8 and any other electronic device 88 to interface with one or more bionic limbs, prosthetics, osseointegration limbs implants and orthotics, including exoskeletons Wherein, the signal acquisition (AI) program for sensory and the signal acquisition (AI) for motion are interchangeable between the physical world and the digital word enabling the user to multitask in the physical world and in the digital world. In this control environment the user can pre-program scalable functions within any portable digital display as the the video and audio are recorded and played on demand for precision signal acquisition programming between all computing devices. Furthermore, the user without the (TMI), (TSI), bionic limbs, prosthetics, osseointegration limbs implants and orthotics, benefits greatly from the utilization of the exoskeleton and wearables operational with signal acquisition from imagined, performed or partially performed EMG, EEG and or EcoG signal acquisition from neuromuscular, sensory region, gesture, touch points, grip, motion, etc.

It is important to mention that the invention includes haptic technology with or without the hand support device 10. Preferably, with the hand support device 10 is a haptic system enable the user to have posture support throughout the upper extremity and lower extremity lumbar spine region while operating any smart computing electronic device 88, headset and or computer device 8 that utilizes EMG, EEG, and or EcoG signal acquisition, including artificial intelligence with or without quantum technology through the hand support device 10, wherein the haptic technology is provided from or through the hand support device 10.

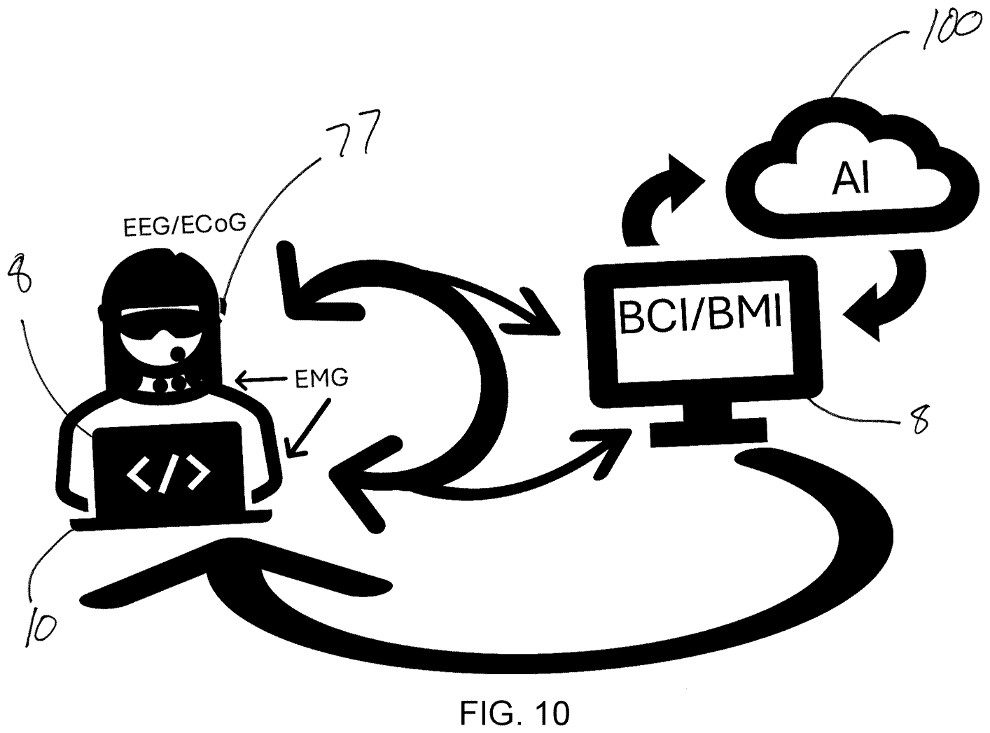

Referring to FIG. 10, in principle, any type of neurological impulse signal could be used to operate with Brain Computer Interface and Brain Machine Interface (BCI/BMI) systems or similar systems etc., wherein a Electroencephalogram (EEG), Magnetoencephalography (MEG), Electrocorticography (EcoG), any Brain Implants (Intracortical Microelectrodes), and etc.. For example, time-trigger EEG or ECoG response amplitudes and latencies, power within specific EEG or ECoG frequency bands or firing rates of individuals cortical neurons. Additionally, upper and lower extremities neuromuscular signals can be utilized, wherein the signal acquisition from Electromyography (EMG), Acceleromyography (AMG), Mechanomyography (MMG), and etc., are converted into digital signals. In other words, the sensors on a (AR/MR/XR/VR) headset are the means for acquiring the signal and measuring the signal of the brain in relation to signal acquisition on the head/scalp through the (EEG), or by the sensors (EcoG) in the skull, or by the sensor (neural implant) in the brain, or any type of signal measurement using a particular sensor modality system. Alternatively, any signal acquisition being produce analyzed and recorded for inputting from a biological hand 7′ or a non-biological limb 7, through or with the use of a hand support device 10 and interfaced with any smart computing electronic device 88 or not connected.

Brain signal acquisition can be formulated from (EEG/ECoG) from neuronal electrical activity in the brain, in relation to any extremities from the arms, neck or legs with a dermis connector sensor modality for signal acquisition, wherein the signal acquisition comes from the (EEG/ECoG) and/or for the (EEG) are amplified or reduced to levels appropriate for electronic processing, including filters for removing electrical noises and for operational through the BCI apparatus or any other similar device, independent or built-in, within the hand support device 10. Also, all signal acquisitions required mentioned above are for the “Feature Extraction” of the processes of analyzing the digital signals. In other words, to differentiate relevant signals characteristics (type of signal and features in the signals, in relation to the final intent) from a biological content and to express them in a compressed form suitable for translation into output commands. Additionally, the resulting signals are then delivered to the “Feature Translation” algorithm, for example the artificial intelligence module built-in, or in the cloud server 100, converts the signal features into the appropriated commands for the hand support device 10 (Device Output). In other words, the “Feature Translation” algorithm provides the translation for the digital functions of the, smart glasses/goggle VR, AR, MR, XR (AI) program display medium headset 77, computer electronic device 8 or any other electronic device 88 (Devices Output) such as letters selections, cursors control, gestures selection pairing program and/or program function, sideline element movement, etc. Through the hand support device 10 the imagined, performed or partially performed EMG, EEG and or EcoG signal acquisition has the necessary independents to isolation for each signal acquisition production, analyzation and recording for scalable programing individually and collectively and the ability to combine more than one signal acquisition, from the smart glasses/goggle VR, AR, MR, XR (AI) program display medium headset 77, or any other device. In other words, through the hand support device 10 the user recorders and creates a robust scalable order and system of simple signals, combination signals and complex signals, that are independent or in combination identified by the artificial intelligence program and recorded for utilization with the smart glasses/goggle VR, AR, MR, XR (AI) program display medium headset 77, computer electronic device 8 or any other computer 88, including from any bionic limbs, prosthetics, osseointegration limbs implants and orthotics, or exoskeletons systems. In other words, the hand support device 10 is the control system, providing relaxation of all the upper extremity neuromuscular signals and neuronal electrical activity in the brain, this process is vital for the recording of independent, combination or complex signal acquisition and for the creation of scalability signal acquisition for inputting and programing. It is important to mention that the (BCI/BMI) can be operated as an accessory to the hand support device 10, or in combination with the artificial intelligence module within the computer electronic device 8 or built-in within any other electronic device 88, including the smart glasses/goggle VR, AR, MR, XR (AI) program display medium headset 77, including from any bionic limbs, prosthetics, osseointegration limbs implants and orthotics, or exoskeletons systems wearable, in combination with one or more physical processors or independently with processors within the cloud sever 100A.

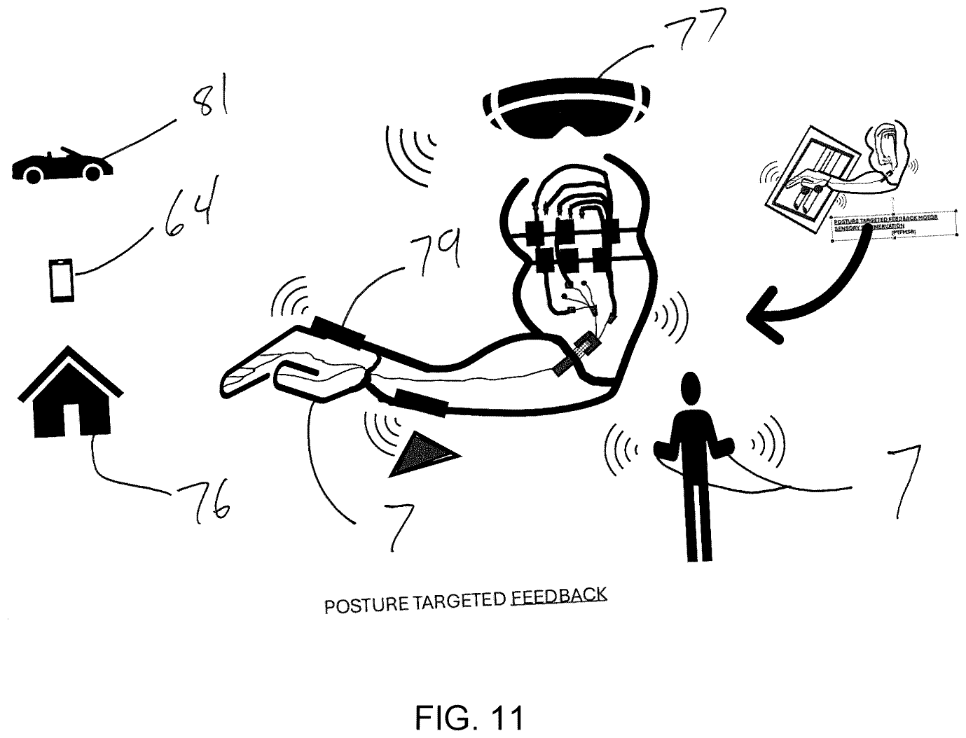

Referring to FIG. 11, the interoperability of inputting systems without the hand support device 10 is embodied as an independent inputting system and as a collection of inputting systems with any smart computing electronic device 88, smart glasses/goggle VR, AR, MR, XR (AI) program display medium headset 77, with one or more computer electronic device 8 within AI/BCI/BMI operational digital systems. In other words, the CPU within the bionic limbs, prosthetics, osseointegration limbs implants and orthotics, including exoskeletons systems are not limited to the mechanical operations of the bionic limbs, prosthetics, osseointegration limbs implants and orthotics, including exoskeletons systems. Furthermore, the prosthetic is a “smart prosthetic”. In other words, the bionic limbs, prosthetics, osseointegration limbs implants and orthotics, including exoskeletons systems are all smart computer systems that have multiple upgrades and operate with smartphones, smart smart glasses/goggle VR, AR, MR, XR (AI) program display medium headset 77, or any other electronic smart devices. The creation of the smart bionic limbs, prosthetics, osseointegration limbs implants and orthotics, including exoskeletons systems, are not just for the operations of the bionic limbs, prosthetics, osseointegration limbs implants and orthotics, including exoskeletons systems. In other words, the independent smartphone 64, smart glasses/goggle VR, AR, MR, XR (AI) program display medium headset 77, or any smart computing electronic device 88 are all redundant. The smart bionic limbs 79, or any electronic prosthetics with or without osseointegration limbs implants including wearables orthotics, and exoskeletons systems will have all the computing capabilities to interface with any smart computing electronic device 88 and to operate an electrical car/hybrid vehicle 81, all the computer applications within a smartphone, all the appliances and amenities within a home, at work and during vacations any fix facility 76.

Referring to FIG. 11, the interoperability of inputting systems without the hand support device 10 is embodied as an independent inputting system and as a collection of inputting systems with any smart computing electronic device 88, smart glasses/goggle VR, AR, MR, XR (AI) program display medium headset 77, with one or more computer electronic device 8 within AI/BCI/BMI operational digital systems. Furthermore, the brain signals (EcoG, EEG), the extremity signals of Electromyography (EMG) and the verbal commands can be used in combination or independently for controlling digital electronic devices. The user may position the electrodes on the anterior portion of the neck for the signal acquisition of the neuromuscular system in relation to speaking with or without voice commands. Furthermore, the type of signal acquisitions (EEG, EcoG, EMG) can come from injured and non-injured body part, such as a brain lesion and/or injured limb. In other words, the artificial intelligence learning capacity from any smart computing electronic device 88, in the smart bionic limbs 79, or any electronic prosthetics with or without osseointegration limbs implants including wearables orthotics, and exoskeletons systems wearables are direct contributions from the hand support device 10.

The significant difference in the present invention is the interoperability of utilizing any neurological signal from a human body, wherein one or more signal acquisitions are measurable from the production of one or more gestures performed or from an imagining gesture, including verbal command or an intended vocalization from a healthy human body and/or from a less than optimal human body. The teachings include method and arrangement for interoperability with various types of signal acquisition unique to the individual from the neuromuscular signals and/or from the brain signals, wherein the signal acquisition is processed and converted to signals that are used for inputting digital or mechanical commands corresponding to an any other computer device 88. Through the hand support device 10 with software/hardware like the (BCI), or equipped with the computer electronic device 8, wirely or wireless, or through a built-in device within the hand support device 10, wherein the computing functions for the conversion of the signal acquisition to the digital base signal are operated online or through the cloud server 100.

The operation of the program of the artificial intelligence module 25 in the hand support device 10, can be implemented in multiple ways as being interfaced with the Brain Computer Interface (BCI) own algorithm system, or as two independent artificial intelligence modules working together, with one for the gesture touch point within the built-in or attachable computer electronic device 8 touch screen of the hand support device 10. The other for the signal acquisition of the imaging gesture or the performed gesture, for the function of the (BCI) to serve in the function of translating and recognizing the desire performed gesture or the imagining gesture, including the performed verbal command or the imagine verbal command, for any form of computerize equipment operations or mechanical robotic machinery interface with the hand support device 10, operating within the usage of the (BCI/BMI) system through a separate device, through a built-in system or through an interoperability of the selected operations in the internet and or through the cloud server 100. It is important to mention that the invention also teaches gestures that are physically performed that are capture recorded, analyzed, and program by the cameras 16 within the hand support device 10, by the headset cameras 74, within the smart glasses/goggle VR, AR, MR, XR (AI) program display medium headset 77 or by any other cameras operational with any other computer device 88 to generate a digital response.

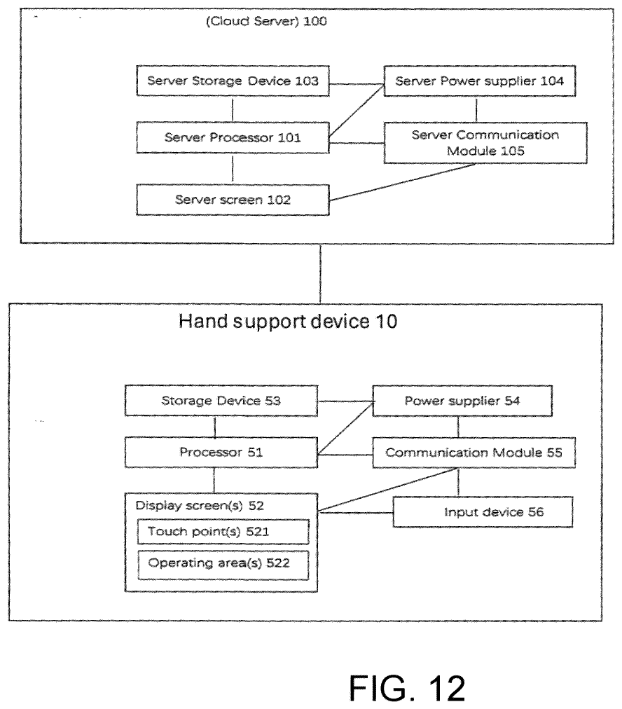

Referring to FIG. 12, the present invention provides a wireless or wirely electrical signal acquisition for operating hand support device 10 through the use of any signal acquisition, like Brain Computer Interface devices, or other similar devices at least consisting of four sequential components: (1) signal acquisition, (2A) feature extraction, (3) feature translation, and (4) device output. These 4 components are controlled by an operating protocol that defines the onset and timing of operation, the details of signal processing, the nature of the device commands, and the oversight of performance. Furthermore, the signal is pairing with a performed gesture and/or paired with an intent gesture, wherein either algorithm system performance can be provided from the hand support device 10 and or from the (BCI) algorithm system or any similar like artificial intelligence computing system online or through the cloud server 100, wirely or wirelessly, through the internet, intranet or from or from other communication servers, wherein signal acquisitions for any body part are used as in any traditional Brain Computer Interface (BCI) system. The present invention further provides that the signal acquisition is paired with the gesture and then the gesture is paired with a program or vice versa. Additionally, the signal acquisition can also be paired or not paired with a program or a gesture, and the gesture can be performed or imaging or partially performed. An algorithm system incorporated in the hand support device 10 is also capable of anticipating the user next imagined, performed or partially performed signal acquisition without the user producing a complete performed gesture or a complete and or imaging gesture, where the information is acquired from data of past performances including any or all performed or not performed gesture with or without paired signal acquisition. Where the commands are controlled and generated by any imagined, performed or partially performed EMG, EEG and or EcoG signal acquisition, from any bionic limbs, prosthetics, osseointegration limbs implants and orthotics, or exoskeletons systems wearable, electronic computer device 88, computer electronic device 8, smart glasses/goggle VR, AR, MR, XR (AI) program display medium headset 77 and or hand support device 10 and are operated to be displayed through one or more display screens, divided by one or more sideline elements 21-23, through the usage of one or more processors 51 operational online, or in a cloud server 100 having a computing processing system with or without the processors 51 in any electronic device 50.

The Brain Computer Interface system is equipped with a built-in algorithm system, attachable or detachable to the hand support device 10, with wired or wireless interface with the analyzable factor module 26 and the artificial intelligence module 25A of any smart computing electronic device 88 for the signal acquisition of gestures, eye motion, retinal movement and/or verbal commands. In other words, any type of human electrical impulse recording during any physiological movements, or any imagine movements, for a physical gesture, a verbal command, a verbal intent, any physical movement, or intended command. Accordingly, in the present invention, the interoperability of Brain Computer Interface (BCI) system or the like can be used for inputting on any smart computing electronic device 88 at the same time or in a timely manner on one or more programs divided by the sideline element 21-23 on the display screen medium.

Furthermore, the present invention provides a system of pairing verbal command with a program or a program function response, through the use of a system like the Brain Computer Interface system with one or more signal acquisitions paired with selected programs or program functions. It is worth mentioning that the verbal function can have more than one words for the desire command to generate the desire response, in the performed vocalization or in the imagine vocalization through the usage of (EEG, EcoG, EMG) signal acquisition for the (BCI), performed within the processors of an electronic hand support device 10, a Brain Computer Interface (BCI), or without processors in the any electronic computing device 88, wherein the signal acquisition information is transmitted to the cloud server 100A and converted to the digital data base for the purpose of operating one or more electronic computing devices 88, as mentioned in the present invention.

Alternatively, the verbal commands operation system can also come from an electromyography (EMG) signal acquisition from the neck voice track neuromuscular system, wherein the signals acquisition is paired with a selected program or program function in a Brain Computer Interface like devices for operating the any electronic computing device 88. In this system the signal acquisition is acquired with audible or without audible vocalization of the verbal command. In other words, the system is not dependent on the audio software to recognize the command. The signal acquisition will require similar processing as earlier mentioned, for the Brain Computer Interface (BCI) system, the major advantage in this system is that the signal acquisition for operating the device is not sound dependent, in other words it will also operate in a noisy environment or without the user producing complete audible vocalization (enunciation). The problem with traditional voice activation programs for verbal commands they require a clean audible sound without noise pollution and in most cases proper enunciation of the selected language, otherwise the verbal command is misunderstood or is non-functional.

Furthermore, Electrooculogram (EOG) signal acquisition for eye movement or the electroretinogram signal acquisition for visual stimuli, these signals can also be process in the same manner as the other signal acquisition wherein processed for the Brain Computer Interface (BCI) devices. Again, the signal acquisition is paired with a selected program or program function, independent or in combination with other signal acquisition paired with other programs or program function, or not paired to the same program, or not paired to a program function. Wherein any signal from the human body can be used to paired with said programs or programs functions, through the use of a Brain Computer Interface like device etc., in addition to the gesture pairing with programs or programs functions in the any electronic computing devices 88, also mention earlier in this invention. The invention teaches the interoperability of signal acquisitions, software and hardware computing systems, etc.

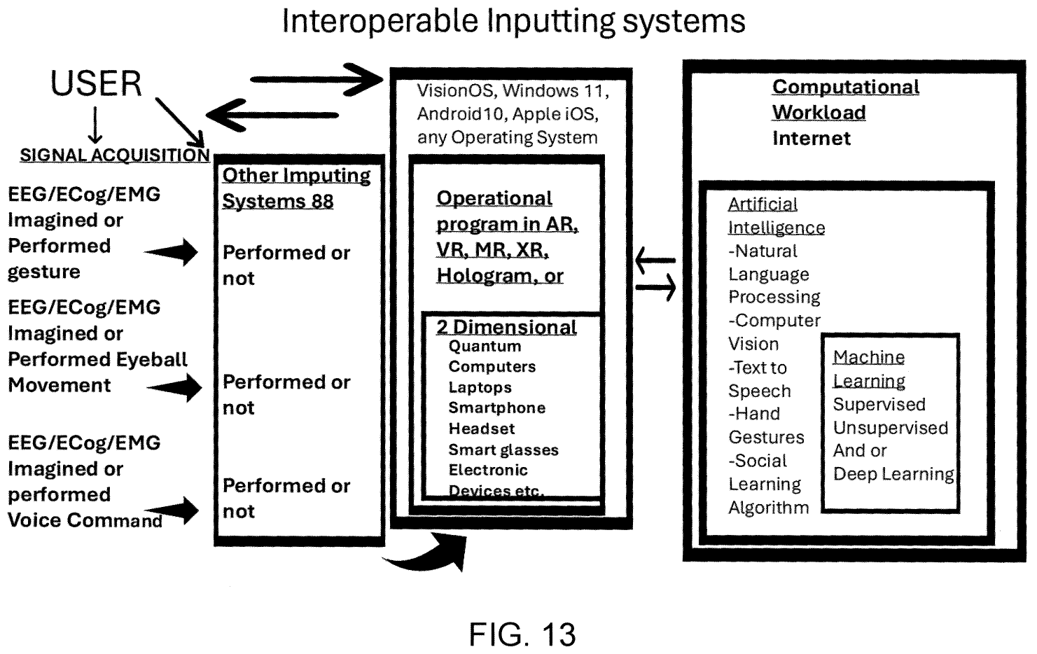

Referring to FIG. 13, the interoperability of performed and imagine signal acquisition with or without the hand support device 10 is illustrated, including smart glasses/goggle VR, AR, MR, XR (AI) program display medium headset 77, laptop, smartphone etc., where the signal acquisition is delivered and or acquired by the hand support device 10, at the same time or in a timely manner in a close loop system, wirely or wireless, through the internet or through the cloud server 100A, wherein the signals are interdependent and or independent on any smart computing electronic device 88 for operating the one or more smart computing electronic device 88, the physical e-mouse 63 and smart glasses/goggle VR, AR, MR, XR (AI) program display medium headset 77, etc. In other words, the interfaced devices will not require the user to provide needlessly repetitive information, including the user gesture selections from the computer electronic device 8 (imagine or performed), command from either the hand activated by the hand support device 10, from the computer electronic device 8 and or from the smart glasses/goggle VR, AR, MR, XR (AI) program display medium headset 77 or any bionic limbs, prosthetics, osseointegration limbs implants and orthotics, or exoskeletons systems wearable, (imagine or performed). Additionally, the line-of-sight fovea eye motion programs (imagine or performed). Alternatively, the user selects the degree and complexity of information shared within any electronic devices, collectively or independently. For example, a personal electronic device will have a robust security system to prevent unwanted usage from uninvited individuals, wherein the gestures, the program selections, where any (signal acquisition) verifications are analyzed for the type of gesture performed or imagine, with or without the touch point, with or without infrared base gestures with or without electromagnetic sensors on one or more electronic computer device 88 with one or more physical display screens from any electronic smart device type computer electronic device 8 or virtual (non-physical) display screen through one or more projecting devices or imaging delivering system such as smart glasses/goggle VR, AR, MR, XR (AI) program display medium headset 77, and being operated by an operation device such as control console, laptop, tablet, smartphone, or hand support device 10 etc. Alternatively, the hand support device 10 may not be a personal electronic device more communal for multiple users with limited customized information to provide and or to receive, in accordance with the current operator.

To individual consumers, who have physical limitation or incapable due to physical fatigue, medical conditions, or lack of interests, may not willing to perform body motions or verbal vocalizations currently required to operate haptic animation surrogate technology in the Virtual Reality environment. According to the preferred embodiment of the present invention, such users have the option to operate in a simulated environment, with or without full body wearables inputting sensors. In other words, the present invention significantly delivers exponentially more inputting flexible options for operating programs, in a more convenient approach to the user selection of electrodes utilization for the desire signal acquisition for operating 2-Dimensions display, 3-Dimensions display, and 4-Dimensional display in any smart glasses/goggle VR, AR, MR, XR (AI) program display medium headset 77. The touch point gestures paired program is able to be operated in combination with the signal acquisition head electrodes or skull implants, electromagnetic sensors, infrared based technology, where the user selects the desire wearables inputting system without interference from the none selected inputting system, and that all inputting systems may also operated together, through the internet, the internet of things (IOT), quantum computer or the cloud server 100A.

It is worth mentioning that the smart hand support device 10′ may also provide the computing power for the smart glasses/goggle VR, AR, MR, XR (AI) program display medium headset 77, just like Brain Computer Interface systems and, furthermore, may acquiring selected signals, receiving selected signals, delivering selected signals and/or processing selected signals from all the signal acquisitions delivered to the smartphone type any electronic computing device 88, to generate a digital response in a close loop system within the smart glasses/goggle VR, AR, MR, XR (AI) program display medium headset apparatus or in any display screen medium. In other words, the smart hand support device 10′ like any other stationary or portable computer may have a compatible attachable, detachable, or built-in amplified that can receive (EMG, EEG, ECoG) signals acquisition from the user where the signals are processed by the smart hand support device 10′ in the same process as in a Brain Computer Interface. Alternatively, smart hand support device 10′, with or without a signal amplifier, may receive a signal acquisition from the user or the process digital signal from the internet, cloud server 100A, or from quantum computing device after that signal acquisition has been processed. For example, the smart hand support device 10′ may receive the signal acquisition, and then the smart hand support device 10′ is the transmission source for delivering the signal acquisition information to the cloud server base Brain Computer Interface program, for the processing of that signal in the cloud server 100A, to a digital base signal and back to the smart hand support device 10′ in a transceiver like simulation.

It is important to mention any smart computing electronic device 88, like smartphone 64, with smart technology portable or fix, wirely or wireless, attachable or detachable to the hand support device 10 and or attachable or detachable to any bionic limbs, prosthetics, osseointegration limbs implants and orthotics, or exoskeletons systems wearable may or may not produce a smart hand support device 10′ and or smart bionic limb, prosthetics, osseointegration limbs implants and orthotics, or exoskeletons systems wearable (non biological limb 7). Also worth mentioning that any smart computing electronic device 88 or similar computing device may also provide the computing power for the smart glasses/goggle VR, AR, MR, XR (AI) program display medium headset 77, just like Brain Computer Interface systems and, furthermore, may acquiring selected signals, receiving selected signals, delivering selected signals, and/or processing selected signals from all the signals acquisitions delivered to the laptop type electronic device, to generate a digital response in a close loop system within the virtual display screen of the smart glasses/goggle VR, AR, MR, XR (AI) program display medium headset 77 apparatus and/or in the physical display screen of the laptop type, smartphone 64 or electronic device 50A or any smart computing electronic device 88′. In other words, the laptop type electronic device, like any other stationary or portable computer may have a compatible attachable, detachable, or built-in amplified means that can receive (EMG, EEG, ECoG) signals from the user, wirely, wirelessly or remotely, where the biological signals are processed by any smart computing electronic device 88 in the same process as in a Brain Computer Interface. Alternatively, the smartphone 64 type electronic device, with or without a signal amplifier, may receive the signal acquisition from the user and from the cloud server 100A as process digital signal, or from quantum computing device, after that original signal acquisition has been processed. For example, the laptop type electronic device/smartphone 64 may first receive the signal acquisition, where the laptop type any smart computing electronic device 88 is the transmission source for delivering the signal acquisition information to the cloud server base Brain Computer Interface program of the cloud server 100A, for the processing of the original signal acquisition into the digital base signal and that, once the digital base signal is produced, that signal is returned back to any smart computing electronic device 88 in a transceiver like simulation to generate a response on the display screen or displayed to other electronic computing devices 8.

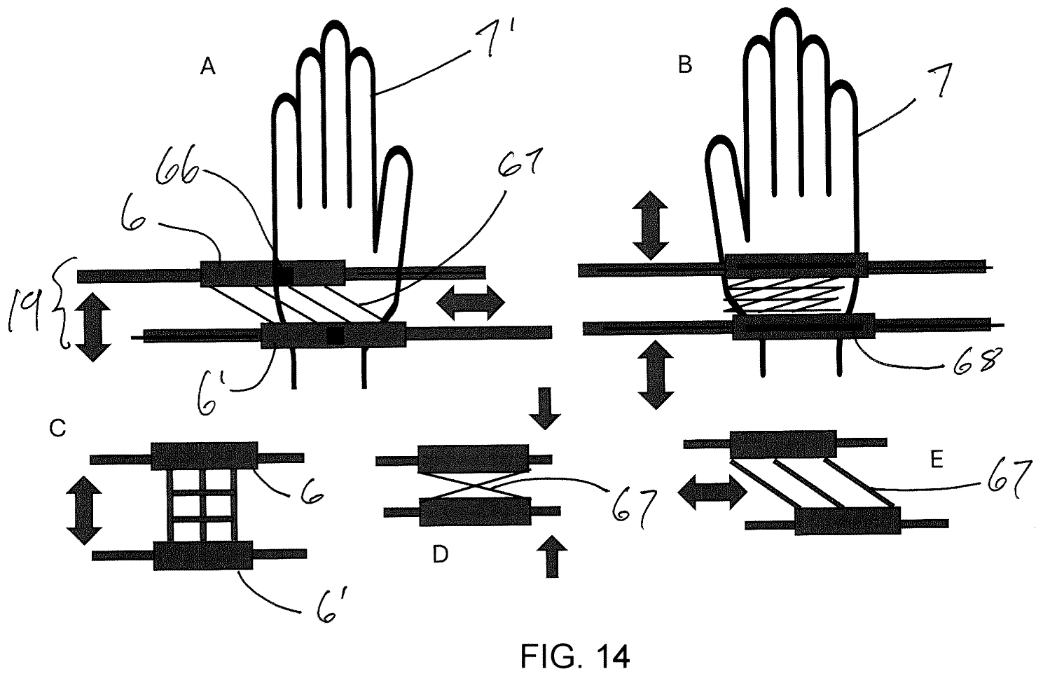

Referring to FIG. 14, the hand support device 10 sleeves 6 are able to operate as one unit, one unit produces a pair for each biological hand 7′ or for each non-biological hand 7. In other words, the non-biological hand 7 and the biological hand 7′ are provided adjustable support for ergonomic upper extremity posture alignment. Additionally, the sleeves 6 are provided with various shapes and pressure absorbing material 56 including various sensors 68 and or various physical interface sensor 66 for interfacing with the hand support device 10 with smart bionic limbs, smart prosthetics, with or without osseointegration limbs implants and smart orthotics, including smart exoskeletons systems. It is important to mention, that the one or more support rods 19 have built in tracks, wire or electrical guide to provide the physical interface with the smart or non-smart bionic limbs, prosthetics, with or without osseointegration limbs implants and orthotics, including exoskeletons systems. It is important to mention that the smart or non-smart hand support device 10 can interface wirelessly with the non-smart or the smart bionic limbs, prosthetics, with or without osseointegration limbs implants, orthotics, including exoskeletons systems. The pair of sleeves for each hand are held together by the sleeve 67 bridge and together function as one unit. It is important to mention that the sleeve bridge 67 allows for quick support and for proper pre-positioning of the sleeves 6 in relation to the user's comfort with or without bionic limbs, prosthetics, osseointegration limbs implants and orthotics, including exoskeletons systems.

As shown in portion B of FIG. 14, both sleeves are aligned equally in the horizontal plane in relation to the perpendicular hand in the neutral position allowing the hand fat pad adipose structure of the palmar fascia 70 to rest between the proximal sleeve 6′ and distal sleeve 6 directly across and over the one or more support bar 19. In this example the user is not concerned for additional thumb movement that may or may not be restricted from surface contact with the distal sleeve 6 pressure material 71. FIG. 14A illustrates both sleeves not aligned equally in the horizontal plane, where the most distal sleeve 6 is more ulnar position in relation to the hand, and the most proximal sleeve is within the center in the neutral position in relation to the hand allowing the hand fat pad adipose structure of the palmar fascia 70 to rest between the proximal sleeve 6′ and the distal sleeve 6. Furthermore, by moving the distal sleeve more toward the ulnar in relation to the hand that provides the thumb more mobility by reducing contact surface from the sleeve and its pressure material 71. At the same time, the corresponding hand continues to have hand support from the corresponding one or more support rods 19 of the hand support device 10, for one or more hands with or without bionic limbs, prosthetics, with or without osseointegration limbs implants, orthotics, including exoskeletons.

A shown in portion C of FIG. 14, the proximal sleeve 6′ and distal sleeve 6 are aligned equally in the horizontal plane in relation to the perpendicular future placement of the hand in the neutral position allowing the hand fat pad adipose structure of the palmar fascia 70 to rest between the proximal sleeve 6′ and distal sleeve 6 directly over the sleeve bridge 67, the sleeve bridge 67 keeps both the proximal sleeve 6′ and distal sleeves 6 together as one unit for each hand with or without bionic limbs, prosthetics, with or without osseointegration limbs implants, orthotics, including exoskeleton systems. FIG. 14D illustrates the proximal sleeve and distal sleeves are aligned equally in the horizontal plane, with a reduction in spacing between the proximal sleeve and the distal sleeve. In other words, the sleeve bridge material and structural design of the sleeve bridge 67 is collapsable allowing for a reduction in distance between the proximal sleeve 6′ and the distal sleeve 6, including the reduction of the inner space in between, in relation to the perpendicular future placement of the hand in the neutral position allowing the hand fat pad adipose structure of the palmar fascia 70 to rest between the proximal sleeve 6′ and the distal sleeve 6. FIG. 14E illustrates the proximal sleeve 6′ and the distal sleeve 6 are not aligned equally in the horizontal plane, with a reduction in spacing or without a reduction in spacing, between the proximal sleeve and the distal sleeve. The invention teaches various combinations and degrees of separation between the two or more support bars 19 and between the one or more proximal sleeves 6′ and distal sleeves 6. It is important to mention that the sleeves support system can come in various shapes and sizes, wherein the illustration was the stander requirements and furthermore through the sleeves the hand support device can provide medial therapeutic modalities. Additionally, through the wireless or wirely independent electrodes the same therapeutic modalities can be provided to other body parts or different therapeutic modalities can be provided like vagal nerve stimulation in conjunction with other medications for stoke recovery patients. Furthermore, any operation with the hand support device components, medically, therapeutic, military, recreational are digital compatible and mechanically compatible with any smart glasses/goggle VR, AR, MR, XR (AI) program display medium headset technology or the likes.

What is claimed is:

1. A hand support device for operating an input apparatus, comprising:
a hand support structure having at least one bar hand support arranged to one or more hands of a user or a prosthetic limb for ergonomic support during input operation;
at least one sleeve slidably mounted on the bar hand support and including at least one interface sensor configured for detecting contact, pressure, or signal input from the user, the bionic limb, or the prosthetic limb; and
a biological signal acquisition system disposed within the hand support device and configured to receive one or more of an electromyographic (EMG), electroencephalographic (EEG), or electrocorticographic (ECoG) signal.

2. The hand support device, as recited in claim 1, further comprising:
a processor operatively coupled with the biological signal acquisition system to generate a digital control command corresponding to an imagined, performed, or partially performed gesture; and
an interface circuit configured to wirely or wirelessly transmit the digital control command to at least one electronic device.

3. The hand support device, as recited in claim 2, wherein the hand support device is configured to communicate with one or more bionic limbs, prosthetics, osseointegration limb implants, orthotics, or exoskeletons, and to interchange biological signal acquisition between the user and the external devices.

4. The hand support device, as recited in claim 2, wherein the processor activates a dormant interface to temporarily suspend or limit physical movement of the one or more bionic limbs, prosthetics, osseointegration limb implants, orthotics, or exoskeletons while maintaining the signal-acquisition function.

5. The hand support device, as recited in claim 2, further comprising a charging battery configured to wirely or wirelessly supply electrical power to the at least one electronic device, selected from a group consisting of computer electronic device, smart-glasses, goggle headset, and the one or more bionic limbs, prosthetics, osseointegration limb implants, orthotics, or exoskeletons, wherein the charging battery is configured for recharging from at least one of a wall outlet, solar panel, or external power generator.

6. The hand support device, as recited in claim 2, further comprising a microphone/speaker configured to receive a voice command and to transmit an audio signal to the processor, a smart-glasses, or a goggle headset.

7. The hand support device, as recited in claim 6, further comprising at least one camera mounted on the hand support device for gesture recognition or sign-language recognition corresponding to an operation of the at least one electronic device.

8. The hand support device, as recited in claim 2, wherein the processor is programmed with an artificial-intelligence inference program configured to interpret the biological signal acquisition for controlling multiple display programs separated by sideline elements on a touch-screen interface.

9. The hand support device, as recited in claim 8, wherein the processor synchronizes the sideline elements with a line-of-sight fovea cursor in a visual device, including but not limited to a smart-glasses and a goggle headset.

10. The hand support device, as recited in claim 2, wherein the hand support structure is foldable or collapsible through one or more folding hinges for portable use.

11. The hand support device, as recited in claim 1, wherein the bar hand support is adjustably mounted by a bar pillar and includes an adjustable bar-support adjuster to vary height or spacing for ergonomic alignment of the upper extremities.

12. The hand support device, as recited in claim 11, wherein the sleeves are paired by a sleeve bridge forming a proximal sleeve and a distal sleeve arranged to receive a palmar fascia region of the user or the prosthetic limb.

13. The hand support device, as recited in claim 1, wherein the interface sensor in the sleeve activates a Brain-Computer Interface (BCI) or Brain-Machine Interface (BMI) function for signal acquisition or control of the at least one electronic device.

14. The hand support device, as recited in claim 13, wherein the BCI/BMI function communicates through the server cloud to record and update biological signal patterns for artificial-intelligence learning.

15. The hand support device, as recited in claim 1, further comprising one or more skin digital sensors positioned on the hand support device to generate secondary sensory feedback corresponding to tactile contact sensed by a non-biological limb.

16. The hand support device, as recited in claim 1, wherein the biological signal acquisition system is configured to pair an EMG signal from a neuromuscular region with an EEG or ECoG signal from the brain to produce a composite control output for multitasking operations.

17. The hand support device, as recited in claim 7, wherein the processor cooperates with the at least one camera, the microphone/speaker, and a smart-glasses/goggle headset to execute gesture, eye-tracking, and voice-command inputs concurrently.

18. The hand support device, as recited in claim 1, further comprising a touch-screen electronic computer attachable or built-in to the hand support device for displaying multiple programs operated simultaneously by biological or non-biological hands.

19. The hand support device, as recited in claim 1, wherein the hand support device is operable to interface simultaneously with the biological hand and the non-biological limb such that the biological hand controls a first display program and the non-biological limb controls a second display program.

20. The hand support device, as recited in claim 2, wherein the processor, biological signal acquisition system, and interface circuit collectively form an integrated artificial-intelligence, brain-computer-interface, and ergonomic-support system configured to operate with or without the user's bionic limbs, prosthetics, osseointegration limb implants, orthotics, or exoskeletons.
